(19) United States
(12) Patent Application Publication
(10) Pub. No.: US 2026/0265280 A1
(43) Pub. Date: Sep. 10, 2026

(54) Prevention/Treatment of Ischemic Reperfusion Injury

(71) Applicant: Rev Therapeutics, Del Mar, CA (US)

(72) Inventors: Yoshi Satoh, Del Mar, CA (US); Kurt Jarnagin, Del Mar, CA (US); Huisheng Xu, Del Mar, CA (US); Xiaohu Zhang, Del Mar, CA (US); Ning Jia; Elizabeth Squiers, Del Mar, CA (US)

(21) Appl. No.: 19/471,940

(22) PCT Filed: Apr. 2, 2024
(86) PCT No.: PCT/US2024/022644

§ 371 Date: Oct. 2, 2025

Related U.S. Application Data
(60) Provisional application No. 63/494,137, filed on Apr. 4, 2023.

(51) Int. Cl. C07F 9/6561; A61K 31/52; A61K 31/675; A61P 9/10; A61P 13/12; C07D 473/32

(52) U.S. Cl. CPC C07F 9/65616; A61K 31/52; A61K 31/675; A61P 9/10; A61P 13/12; C07D 473/32

(57) ABSTRACT

This invention provides, among other things, compounds useful for treating and/or preventing an injury before, during, or after a surgical procedure.

### CROSS-REFERENCE TO RELATED APPLICATION

Pursuant to 35 U.S.C. § 119(e), this application claims priority to the filing date of U.S. Provisional Patent Application Ser. No. 63/494,137 filed Apr. 4, 2023, the disclosure of which is herein incorporated by reference in its entirety.

### BACKGROUND OF THE INVENTION

Before or during certain surgical procedures, blood flow is reduced to certain organs and/or tissues. Blood flow is then increased during or after the surgical procedure. This increase in blood flow can result in reperfusion injuries (characterized by inflammation, apoptosis, necrosis, and fibrosis) to certain organs and/or tissues.

An orally-active compound which could be useful to treat or prevent such injuries is CC-930:

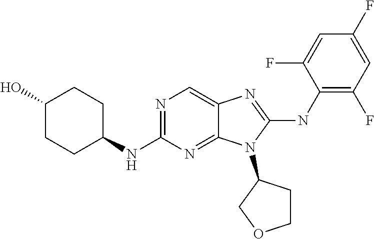 [SMILES: O[C@H]1CC[C@H](Nc2ncc3nc(Nc4c(F)cc(F)cc4F)n([C@H]4CCOC4)c3n2)CC1]

However, subjects undergoing a surgical procedure are unconscious or otherwise unable to take medication orally. Intravenous (IV) administration of the medication would therefore be important. However, CC-930 is only sparingly soluble in common IV formulations (0.06063 mg/mL in phosphate buffered saline, for example). This lack of solubility severely limits its ability to be used in a surgical setting.

There is a need therefore for compounds which are: a) soluble in TV formulations and b) readily converted to CC-930 once in the subject. The present invention addresses these and other needs.

### SUMMARY OF THE INVENTION

The invention provides compounds described herein or salts or tautomers or isomers thereof, pharmaceutical formulations comprising such compounds, and methods of using the same to treat and/or prevent an injury before, during, or after a surgical procedure to a subject, such as a human.

### BRIEF DESCRIPTION OF THE FIGURES

FIG. 1 presents data showing the effect of treatment with compound Q8 on kidney dysfunction, assessed by plasma creatinine levels, measured at 1 day and 4 days post-lesion. Kidney function as assessed by plasma creatinine is restored to near normal on Day 1 by treatment with Compound Q8. Rat two kidney warm short ischemia model. IRI is associated with serum creatinine elevation of 4.75 fold* and is reduced by 87-88% by all treatments vs. 1D vehicle.

FIG. 2 presents data on the effect to kidney tubular damage by treatment with compound Q8 as measured at 7 days post-lesion. Compound Q8 reduces tubular damage by 65% on Day 7.

FIG. 3 presents rat bilateral kidney warm short ischemia model data. Compound Q8 reduces fibrosis component Coll histological staining by 73% on D21.

### DETAILED DESCRIPTION OF THE INVENTION

### I. Definitions and Abbreviations

In order that the application may be more completely understood, several definitions are set forth below. Such definitions are meant to encompass grammatical equivalents.

The term “about” in relation to a reference numerical value can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

The terms “a,” “an,” “the” and similar referents used in the context of describing the disclosure (especially in the context of the following claims) are to be construed to cover both the singular and the plural, unless otherwise indicated herein or clearly contradicted by context.

The following abbreviations have been used: Ac is acetyl; AcOH is acetic acid; ACTBr is cetyltrimethylammonium bromide; AIBN is azobisisobutyronitrile or 2,2 azobisisobutyronitrile; aq. is aqueous; Ar is aryl; B₂pin₂ is bis(pinacolato)diboron; Bn is, in general, benzyl [see Cbz for one example of an exception]; (BnS)₂ is benzyl disulfide; BnSH is benzyl thiol or benzyl mercaptan; BnBr is benzyl bromide; Boc is tert-butoxy carbonyl; Boc₂O is di-tert-butyl dicarbonate; Bz is, in general, benzoyl; BzOOH is benzoyl peroxide; Cbz or Z is benzyloxycarbonyl or carboxybenzyl; Cs₂CO₃ is cesium carbonate; CSA is camphor sulfonic acid; CTAB is cetyltrimethylammonium bromide; Cy is cyclohexyl; DABCO is 1,4-diazabicyclo[2.2.2]octane; DCM is dichloromethane or methylene chloride; DHP is dihydropyran; DIAD is diisopropyl azodicarboxylate; DIEA or DIPEA is N,N-diisopropylethylamine; DMAP is 4-(dimethylamino)pyridine; DME is 1,2-dimethoxyethane; DMF is N,N-dimethylformamide; DMSO is dimethylsulfoxide; equiv or eq. is equivalent; EtOAc is ethyl acetate; EtOH is ethanol; Et₂O is diethyl ether; EDCI is N-(3-dimethylaminopropyl)-N′-ethylcarbodiimide hydrochloride; ELS is evaporative light scattering; equiv or eq is equivalent; h is hours; HATU is O-(7-azabenzotriazol-1-yl)-N,N,N′,N′-tetramethyluronium hexafluorophosphate; HOBt is N-hydroxybenzotriazole; HCl is hydrochloric acid; HPLC is high pressure liquid chromatography; ISCO Companion is automated flash chromatography equipment with fraction analysis by UV absorption available from Presearch; KOAc or AcOK is potassium acetate; K₂CO₃ is potassium carbonate; LiAlH₄ or LAH is lithium aluminum hydride; LDA is lithium diisopropylamide; LHMDS is lithium bis(trimethylsilyl) amide; KHMDS is potassium bis(trimethylsilyl) amide; LiOH is lithium hydroxide; m-CPBA is 3-chloroperoxybenzoic acid; MeCN or ACN is methyl cyanide or cyanomethane or ethanenitrile or acetonitrile which are all names for the same compound; MeOH is methanol; MgSO₄ is magnesium sulfate; mins or min is minutes; Mp or MP is melting point; NaCNBH₃ is sodium cyanoborohydride; NaOH is sodium hydroxide; Na₂SO₄ is sodium sulfate; NBS is N-bromosuccinimide; NH₄Cl is ammonium chloride; NIS is N-iodosuccinimide; N₂ is nitrogen; NMM is N-methylmorpholine; n-BuLi is n-butyllithium; overnight is O/N; PdCl₂(pddf) is 1,1′-Bis(diphenylphosphino) ferrocene]dichloropalladium(II); Pd/C is the catalyst known as palladium on carbon; Pd₂(dba)₃ is an organometallic catalyst known as tris(dibenzylideneacetone) dipalladium(0); Ra Ni or Raney Ni is Raney nickel; Ph is phenyl; PMB is p-methoxybenzyl; PrOH is 1-propanol; iPrOH is 2-propanol; POCl₃ is phosphorus chloride oxide; PTSA is para-toluene sulfonic acid; Pyr. or Pyr or Py as used herein means pyridine; RT or rt or r.t. is room temperature; sat. is saturated; Si-amine or Si—NH₂ is amino-functionalized silica, available from SiliCycle; Si-pyr is pyridyl-functionalized silica, available from SiliCycle; TEA or Et₃N is triethylamine; TFA is trifluoroacetic acid; Tf₂O is trifluoromethanesulfonic anhydride; THF is tetrahydrofuran; TFAA is trifluoroacetic anhydride; THP is tetrahydropyranyl; TMSI is trimethylsilyl iodide; H₂O is water; diNO₂PhSO₂Cl is dinitrophenyl sulfonyl chloride; 3-F-4-NO₂—PhSO₂Cl is 3-fluoro-4-nitrophenylsulfonyl chloride; 2-MeO-4-NO₂—PhSO₂Cl is 2-methoxy-4-nitrophenylsulfonyl chloride; and (EtO)₂POCH₂COOEt is a triethylester of phosphonoacetic acid known as triethyl phosphonoacetate.

“Compound of the invention,” as used herein refers to the compounds discussed herein, salts (e.g. pharmaceutically acceptable salts), solvates and hydrates of these compounds.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “poly” as used herein means at least 2. For example, a polyvalent metal ion is a metal ion having a valency of at least 2.

“Moiety” refers to a radical of a molecule that is attached to the remainder of the molecule.

The symbol [symbol] , whether utilized as a bond or displayed perpendicular to a bond, indicates the point at which the displayed moiety is attached to the remainder of the molecule.

The term “alkyl,” by itself or as part of another substituent, means, unless otherwise stated, a straight or branched chain, or combination thereof, which may be fully saturated, mono-or polyunsaturated and can include di-and multivalent radicals, having the number of carbon atoms designated (i.e. C₁-C₁₀ means one to ten carbons).

In some embodiments, the term “alkyl” means a straight or branched chain, or combinations thereof, which may be fully saturated, mono-or polyunsaturated and can include di-and multivalent radicals. Examples of saturated hydrocarbon radicals include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1-and 3-propynyl, 3-butynyl, and the higher homologs and isomers.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the invention. A “lower alkyl” or “lower alkylene” is a shorter chain alkyl or alkylene group, generally having eight or fewer carbon atoms.

The term “alkenylene” by itself or as part of another substituent means a divalent radical derived from an alkene.

The term “cycloalkylene” by itself or as part of another substituent means a divalent radical derived from a cycloalkane.

The term “heteroalkylene” by itself or as part of another substituent means a divalent radical derived from an heteroalkane.

The term “heterocycloalkylene” by itself or as part of another substituent means a divalent radical derived from an heterocycloalkane.

The term “arylene” by itself or as part of another substituent means a divalent radical derived from an aryl.

The term “heteroarylene” by itself or as part of another substituent means a divalent radical derived from heteroaryl.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom. In some embodiments, the term “heteroalkyl,” by itself or in combination with another term, means a stable straight or branched chain, or combinations thereof, consisting of the stated number of carbon atoms and at least one heteroatom. In an exemplary embodiment, the heteroatoms can be selected from the group consisting of B, O, N and S, and wherein the nitrogen and sulfur atoms may optionally be oxidized and the nitrogen heteroatom may optionally be quaternized.

The heteroatom(s) B, O, N and S may be placed at any interior position of the heteroalkyl group or at the position at which the alkyl group is attached to the remainder of the molecule. Examples include, but are not limited to, —OCH₃, —CH₂CH₂OCH₃, —CH₂CH₂NHCH₃, —CH₂CH₂N(CH₃)CH₃, —CH₂CH₂CH₂N(CH₃)CH₃, —CH₂SCH₂CH₃, —CH₂CH₂S(O)CH₃, —CH₂CH₂S(O)₂CH₃, —CH═CHO—CH₃, —CH₂—CH═N—OCH₃, and —CH═CH—N(CH₃)—CH₃. Up to two heteroatoms may be consecutive, such as, for example, —CH₂—NH—OCH₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 2-(4-methylpiperazin-1-yl)ethyl, 3-(4-methylpiperazin-1-yl)propyl, 2-morpholinoethyl, 3-morpholinopropyl, 1-methylazetidin-3-yl, 1-ethylazetidin-3-yl, 1-isopropylazetidin-3-yl, 1-methylpiperidin-4-yl, 1-ethylpiperidin-4-yl or, 1-isopropylpiperidin-4-yl, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 2-fluoroethyl, 2,2-difluoroethyl, 2-chloroethyl, 3-chloropropyl, 4-chlorobutyl, 3-bromopropyl, and the like.

The term “aryl” means, unless otherwise stated, a polyunsaturated, aromatic, substituent that can be a single ring or multiple rings (preferably from 1 or 2 or 3 rings), which are fused together or linked covalently. The term “heteroaryl” refers to aryl groups (or rings) that contain from one to four heteroatoms. In an exemplary embodiment, the heteroatom is selected from B, N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —R′, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR′′″—C(NR′R″R′″)═NR′″, —NR′″—C(NR′R″)═NR′″, —S(O)R′, —S(O)₂R′, —S(O)₂NR′R″, —NR″SO₂R′, —CN, —NO₂, —N₃, —CH(Ph)₂, fluoro(C₁-C₄)alkoxy, and fluoro(C₁-C₄)alkyl, in a number ranging from zero to (2m′+1), where m′ is the total number of carbon atoms in such radical. R′, R″, R′″, R″″ and R′″″ each preferably independently refer to hydrogen, substituted or unsubstituted haloalkyl, substituted or unsubstituted heteroalkyl, substituted or unsubstituted heterocycloalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″, R″″ and R′″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: —R′, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR′″″—C(NR′R″R′″)═NR″″, —NR″″—C(NR′R″)═NR′″, —S(O)R′, —S(O)₂R′, —S(O)₂NR′R″, —NR″SO₂R′, —CN, —NO₂, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, R″″ and R′″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″, R′″″ and R′ groups when more than one of these groups is present.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted C₁ or C₂ or C₃ or C₄ or C₅ or C₆ alkyl.

“Ring” as used herein, means a substituted or unsubstituted cycloalkyl, substituted or unsubstituted heterocycloalkyl, substituted or unsubstituted aryl, or substituted or unsubstituted heteroaryl. A ring includes fused ring moieties. The number of atoms in a ring is typically defined by the number of members in the ring. For example, a “5-to 7-membered ring” means there are 5 or 6 or 7 atoms in the encircling arrangement. Unless otherwise specified, the ring optionally includes a heteroatom. Thus, the term “5 to 7-membered ring” or “5 or 6 or 7 membered ring” includes, for example phenyl, pyridinyl and piperidinyl. The term “5 to 7-membered heterocycloalkyl ring” “5 or 6 or 7-membered heterocycloalkyl ring”, on the other hand, would include pyridinyl and piperidinyl, but not phenyl. The term “ring” further includes a ring system comprising more than one “ring”, wherein each “ring” is independently defined as above.

As used herein, the term “heteroatom” includes atoms other than carbon (C) and hydrogen (H). Examples include oxygen (O), nitrogen (N) sulfur (S), silicon (Si), germanium (Ge), and aluminum (Al).

The term “leaving group” means a functional group or atom which can be displaced by another functional group or atom in a substitution reaction, such as a nucleophilic substitution reaction. By way of example, representative leaving groups include triflate, chloro, bromo and iodo groups; sulfonic ester groups, such as mesylate, tosylate, brosylate, nosylate and the like; and acyloxy groups, such as acetoxy, trifluoroacetoxy and the like.

The symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, substituted or unsubstituted cycloalkyl and substituted or unsubstituted heterocycloalkyl groups.

By “effective” amount of a drug, formulation, or permeant is meant a sufficient amount of an active agent to provide the desired local or systemic effect. A “Topically effective,” “pharmaceutically effective,” or “therapeutically effective” amount refers to the amount of drug needed to effect the desired therapeutic result.

The term “pharmaceutically acceptable salt” is meant to include a salt of a compound of the invention which is prepared with relatively nontoxic acids or bases, depending on the particular substituents found on the compounds described herein. When compounds of the invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino (such as choline or diethylamine or amino acids such as d-arginine, 1-arginine, d-lysine, or 1-lysine), or magnesium salt, or a similar salt. When compounds of the invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science 66: 1-19 (1977)). Certain specific compounds of the invention contain both basic and acidic functionalities that allow the compounds to be converted into either base or acid addition salts.

The neutral forms of the compounds are preferably regenerated by contacting the salt with a base or acid and isolating the parent compounds in the conventional manner. The parent form of the compound differs from the various salt forms in certain physical properties, such as solubility in polar solvents.

Certain compounds of the invention can exist in unsolvated forms as well as solvated forms, including hydrated forms. In general, the solvated forms are equivalent to unsolvated forms and are encompassed within the scope of the invention. Certain compounds of the invention may exist in multiple crystalline or amorphous forms.

Certain compounds of the invention possess asymmetric carbon atoms (optical centers) or double bonds; the racemates, diastereomers, geometric isomers and individual isomers are encompassed within the scope of the invention. The graphic representations of racemic, ambiscalemic and scalemic or enantiomerically pure compounds used herein are taken from Maehr, J. Chem. Ed. 1985, 62: 114-120. Solid and broken wedges are used to denote the absolute configuration of a stereocenter unless otherwise noted. When the compounds described herein contain olefinic double bonds or other centers of geometric asymmetry, and unless specified otherwise, it is intended that the compounds include both E and Z geometric isomers. Likewise, all tautomeric forms are included.

Compounds of the invention can exist in particular geometric or stereoisomeric forms. The invention contemplates all such compounds, including cis- and trans-isomers, (−)-and (+)-enantiomers, (R)-and (S)-enantiomers, diastereomers, (D)-isomers, (L)-isomers, the racemic mixtures thereof, and other mixtures thereof, such as enantiomerically or diastereomerically enriched mixtures, as falling within the scope of the invention. Additional asymmetric carbon atoms can be present in a substituent such as an alkyl group. All such isomers, as well as mixtures thereof, are intended to be included in this invention.

Optically active (R)-and (S)-isomers and d and l isomers can be prepared using chiral synthons or chiral reagents, or resolved using conventional techniques. If, for instance, a particular enantiomer of a compound of the invention is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

The compounds of the invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the invention, whether radioactive or not, are intended to be encompassed within the scope of the invention.

The term “pharmaceutically acceptable carrier” or “pharmaceutically acceptable vehicle” refers to any formulation or carrier medium that provides the appropriate delivery of an effective amount of an active agent as defined herein, does not interfere with the effectiveness of the biological activity of the active agent, and that is sufficiently non-toxic to the subject. Representative carriers include water, oils, both vegetable and mineral, cream bases, lotion bases, ointment bases and the like. These bases include suspending agents, thickeners, penetration enhancers, and the like. Their formulation is well known to those in the art of cosmetics and topical pharmaceuticals. Additional information concerning carriers can be found in Remington: The Science and Practice of Pharmacy, 21st Ed., Lippincott, Williams & Wilkins (2005) which is incorporated herein by reference.

The term “excipients” is conventionally known to mean carriers, diluents and/or vehicles used in formulating drug compositions effective for the desired use.

The terms “effective amount” or a “therapeutically effective amount” of a drug or pharmacologically active agent refers to a nontoxic but sufficient amount of the drug or agent to provide the desired effect. In the oral dosage forms of the present disclosure, an “effective amount” of one active of the combination is the amount of that active that is effective to provide the desired effect when used in combination with the other active of the combination. The amount that is “effective” will vary from subject to subject, depending on the age and general condition of the individual, the particular active agent or agents, and the appropriate “effective” amount in any individual case may be determined by one of ordinary skill in the art using routine experimentation.

The phrases “active ingredient”, “therapeutic agent”, “active”, or “active agent” mean a chemical entity which can be effective in treating a targeted disorder, disease or condition.

The phrase “pharmaceutically acceptable” means moieties or compounds that are, within the scope of medical judgment, suitable for use in humans without causing undesirable biological effects such as undue toxicity, irritation, allergic response, and the like, for example.

“Biological medium,” as used herein refers to both in vitro and in vivo biological milieus. Exemplary in vitro “biological media” include, but are not limited to, cell culture, tissue culture, homogenates, plasma and blood. In vivo applications are generally performed in mammals, preferably humans.

“Inhibiting” and “blocking,” are used interchangeably herein to refer to the partial or full blockade of an enzyme, such as a c-Jun N-terminal kinase (JN K).

Embodiments of the invention also encompass compounds that are poly- or multi-valent species, including, for example, species such as dimers, trimers, tetramers and higher homologs of the compounds of use in the invention or reactive analogues thereof.

### II. Introduction

The invention provides novel compounds. The novel compounds, as well as pharmaceutical formulations containing such compounds are described.

### III. The Compounds

In one aspect, the invention provides a compound or a salt or a tautomer or an isomer thereof. In an exemplary embodiment, the invention is a compound described herein, or a salt or a tautomer or an isomer thereof. In an exemplary embodiment, the invention is a compound according to a formula described herein, or a salt or a tautomer or an isomer thereof.

In one aspect, the invention is a compound of the invention. In an exemplary embodiment, the invention is a compound described herein. In an exemplary embodiment, the invention is a compound according to a formula described herein.

In an exemplary embodiment, the invention is a compound, or a salt or a tautomer or an isomer thereof, having a structure according to formula (I) or (II):

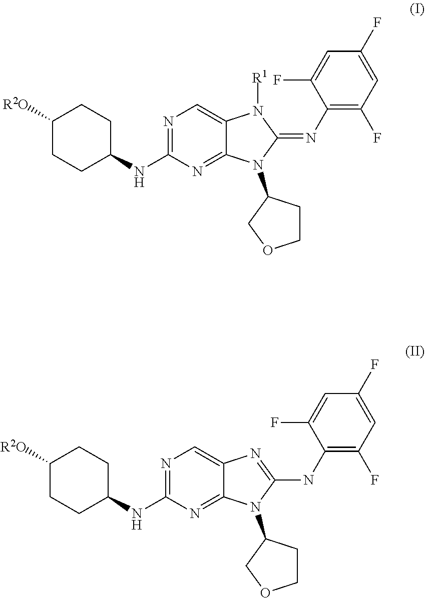

wherein R¹, when present, is C(O)R^{a} or substituted or unsubstituted C₁₋₆ alkylene wherein R^{a} is substituted or unsubstituted C₁₋₆ alkoxy or substituted or unsubstituted C₁₋₆ alkylamino; R² is H or C(O)R^{b} or substituted or unsubstituted phosphate, wherein R^{b} is substituted or unsubstituted C₁₋₆ alkyl or substituted or unsubstituted C₁₋₆ alkoxy or substituted or unsubstituted C₁₋₆ alkylamino, with the proviso that the compound is not

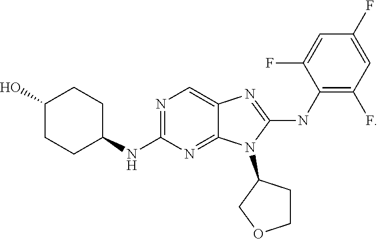 [SMILES: O[C@H]1CC[C@H](Nc2ncc3nc(Nc4c(F)cc(F)cc4F)n([C@H]4CCOC4)c3n2)CC1]

In an exemplary embodiment, the compound, or a salt or a tautomer or an isomer thereof, has a structure according to formula (I):

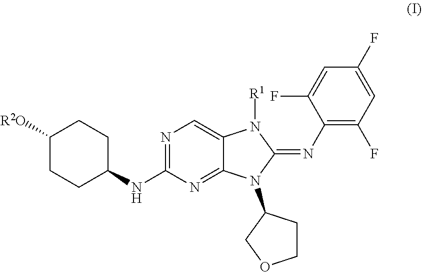

wherein R¹ is C(O)R^{a} or substituted or unsubstituted C₁₋₆ alkylene, wherein R^{a} is substituted or unsubstituted C₁₋₆ alkoxy or substituted or unsubstituted C₁₋₆ alkylamino; R² is H or C(O)R^{b} or substituted or unsubstituted phosphate, wherein R^{b} is substituted or unsubstituted C₁₋₆ alkyl or substituted or unsubstituted C₁₋₆ alkoxy or substituted or unsubstituted C₁₋₆ alkylamino.

In an exemplary embodiment, the compound, or a salt or a tautomer or an isomer thereof, has a structure according to formula (II):

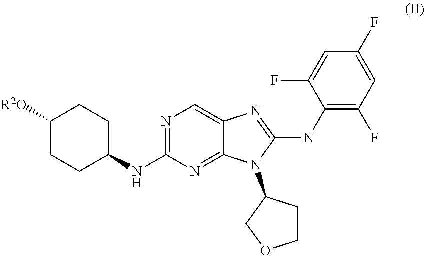

wherein R² is C(O)R^{b} or substituted or unsubstituted phosphate, wherein R^{b} is substituted or unsubstituted C₁₋₆ alkyl or substituted or unsubstituted C₁₋₆ alkoxy or substituted or unsubstituted C₁₋₆ alkylamino.

In an exemplary embodiment, the compound, or a salt or a tautomer or an isomer thereof, has a structure according to formula (I), wherein R¹ is as described herein, and R² is H, C(O)CH₃, C(O)CH₂CH₃, C(O)CH(CH₃)₂, C(O)CH₂OH, C(O)CH₂NH₂, C(O)N(CH₃)₂, C(O)CH(NH₂)CH₃, C(O)OCH₂CH₃, C(O)O(CH₂)₂N(CH₃)₃, C(O)CH₂N(CH₃)₃, or substituted or unsubstituted phosphate. In an exemplary embodiment, the compound, or a salt or a tautomer or an isomer thereof, has a structure according to formula (II), wherein R² is C(O)CH₃, C(O)CH₂CH₃, C(O)CH(CH₃)₂, C(O)CH₂OH, C(O)CH₂NH₂, C(O)N(CH₃)₂, C(O)CH(NH₂)CH₃, C(O)OCH₂CH₃, C(O)O(CH₂)₂N(CH₃)₃, C(O)CH₂N(CH₃)₃, or substituted or unsubstituted phosphate.

In an exemplary embodiment, the compound, or a salt or a tautomer or an isomer thereof, has a structure according to formula (I), wherein R¹ is as described herein, and R² is H.

In an exemplary embodiment, the compound, or a salt or a tautomer or an isomer thereof, has a structure according to formula (I), wherein R¹ is as described herein, and R² is

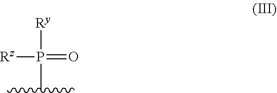

wherein R^{y} and R^{z} are each individually selected from OH, O-phosphate, or C₁₋₆ alkoxy. In an exemplary embodiment, the compound, or a salt or a tautomer or an isomer thereof, has a structure according to formula (II), wherein R² is

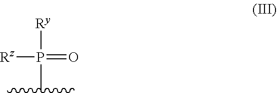

wherein R^{y} and R^{z} are each individually selected from OH, O-phosphate, or C₁₋₆ alkoxy.

In an exemplary embodiment, the compound, or a salt or a tautomer or an isomer thereof, has a structure according to formula (I), wherein R¹ is as described herein, and R² is

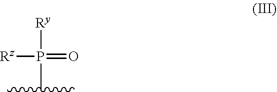

wherein R^{y} and R^{z} are each individually selected from OH, O-phosphate, or C₁₋₆ alkoxy. In an exemplary embodiment, the compound, or a salt or a tautomer thereof, has a structure according to formula (II), wherein R² is

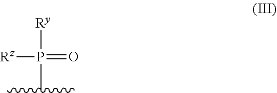

wherein R^{y} and R^{z} are each individually selected from OH, O-phosphate, or C₁₋₆ alkoxy.

In an exemplary embodiment, the compound, or a salt or a tautomer or an isomer thereof, has a structure according to formula (I), wherein R¹ is as described herein, and R² is

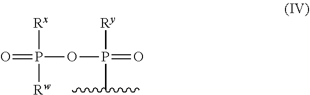

wherein R^{w}, R^{x}, and R^{y} are each individually selected from OH, O-phosphate, or C₁₋₆ alkoxy. In an exemplary embodiment, the compound, or a salt or a tautomer or an isomer thereof, has a structure according to formula (II), wherein R² is

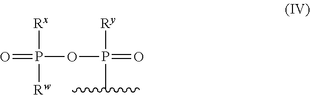

wherein R^{w}, R^{x}, and R^{y} are each individually selected from OH, O-phosphate, or C₁₋₆ alkoxy.

In an exemplary embodiment, the compound, or a salt or a tautomer or an isomer thereof, has a structure according to formula (I), wherein R¹ is as described herein, and wherein R² is

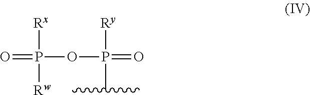

wherein R^{w}, R^{x}, and R^{y} are each individually selected from OH or C₁₋₆ alkoxy. In an exemplary embodiment, the compound, or a salt or a tautomer or an isomer thereof, has a structure according to formula (II), wherein R² is

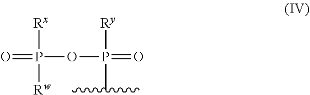

wherein R^{w}, R^{x}, and R^{y} are each individually selected from OH or C₁₋₆ alkoxy.

In an exemplary embodiment, the compound, or a salt or a tautomer or an isomer thereof, has a structure according to formula (I), wherein R¹ is as described herein, and R² is

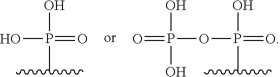 [SMILES: *P(=O)(O)O ; *P(=O)(O)OP(=O)(O)O]

In an exemplary embodiment, the compound, or a salt or a tautomer or an isomer thereof, has a structure according to formula (II), wherein R² is

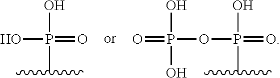 [SMILES: *P(=O)(O)O ; *P(=O)(O)OP(=O)(O)O]

In an exemplary embodiment, the compound, or a salt or a tautomer or an isomer thereof, having a structure according to formula (II).

In an exemplary embodiment, the compound, or a salt or a tautomer or an isomer thereof, has a structure according to formula (I), wherein R² is as described herein, and R¹ has a structure according to formula (V):

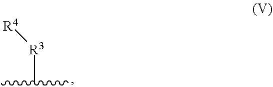

wherein R³ is C═O, CH₂, CH(CH₃), CH(CH₂CH₃), CH(CH(CH₃)₂), or CH(CH₂OH), when R³ is C═O, then R⁴ is unsubstituted C₁₋₆ alkyl, —OR⁵, or NR⁵R⁶, wherein R⁵ and R⁶ are each individually selected from unsubstituted C₁₋₆ alkyl or C₁₋₆ alkyl substituted with a trialkylammonium moiety, when R³ is CH₂, CH(CH₃), CH(CH₂CH₃), CH(CH(CH₃)₂), or CH(CH₂OH), then R⁴ is substituted or unsubstituted phosphate.

In an exemplary embodiment, the compound, or a salt or a tautomer or an isomer thereof, has a structure according to formula (I), wherein R² is as described herein, and R¹ has a structure according to formula (V):

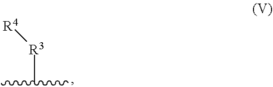

wherein R³ is CH₂, CH(CH₃), CH(CH₂CH₃), CH(CH(CH₃)₂), or CH(CH₂OH), and R⁴ is substituted or unsubstituted phosphate.

In an exemplary embodiment, the compound, or a salt or a tautomer or an isomer thereof, has a structure according to formula (I), wherein R² is as described herein, and R¹ has a structure according to formula (V):

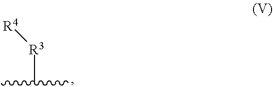

wherein R³ is CH₂, CH(CH₃), CH(CH₂CH₃), CH(CH(CH₃)₂), or CH(CH₂OH), and R⁴ is substituted or unsubstituted phosphate.

In an exemplary embodiment, the compound, or a salt or a tautomer or an isomer thereof, has a structure according to formula (I), wherein R² is as described herein, and R¹ has a structure according to formula (V):

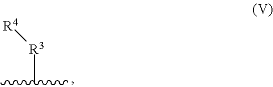

wherein R³ is as described herein, and R⁴ has a structure according to formula (VI)

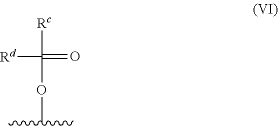

wherein R^{c} and R^{d} are each individually selected from OH, O-phosphate, or C₁₋₆ alkoxy.

In an exemplary embodiment, the compound, or a salt or a tautomer or an isomer thereof, has a structure according to formula (I), wherein R² is as described herein, and R¹ has a structure according to formula (V):

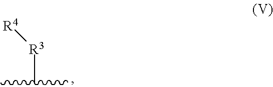

wherein R³ is as described herein, and R⁴ is according to formula (VII)

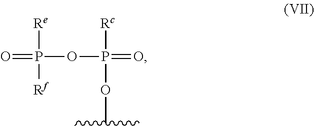

wherein R^{c}, R^{e}, and R^{f} are each individually selected from OH, O-phosphate, or C₁₋₆ alkoxy.

In an exemplary embodiment, the compound, or a salt or a tautomer or an isomer thereof, has a structure according to formula (I), wherein R² is as described herein, and R¹ has a structure according to formula (V):

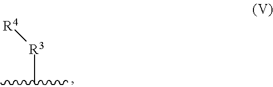

wherein R³ is as described herein, and R⁴ is according to formula (VII)

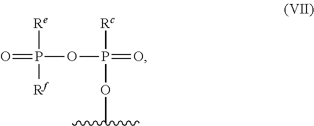

wherein R^{c}, R^{e}, and R^{f} are each individually selected from OH or C₁₋₆ alkoxy.

In an exemplary embodiment, the compound, or a salt or a tautomer or an isomer thereof, has a structure according to formula (I), wherein R² is as described herein, and R¹ has a structure according to formula (V):

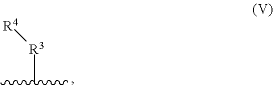

wherein R³ is as described herein, and R⁴ is

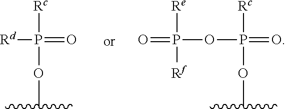

In an exemplary embodiment, the compound, or a salt or a tautomer or an isomer thereof, has a structure according to formula (I), wherein R² is as described herein, and R¹ has a structure according to formula (V):

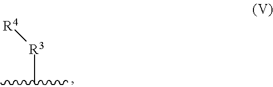

wherein R⁴ is as described herein, and R³ is CH₂.

In an exemplary embodiment, the compound, or a salt or a tautomer or an isomer thereof, having a structure which is

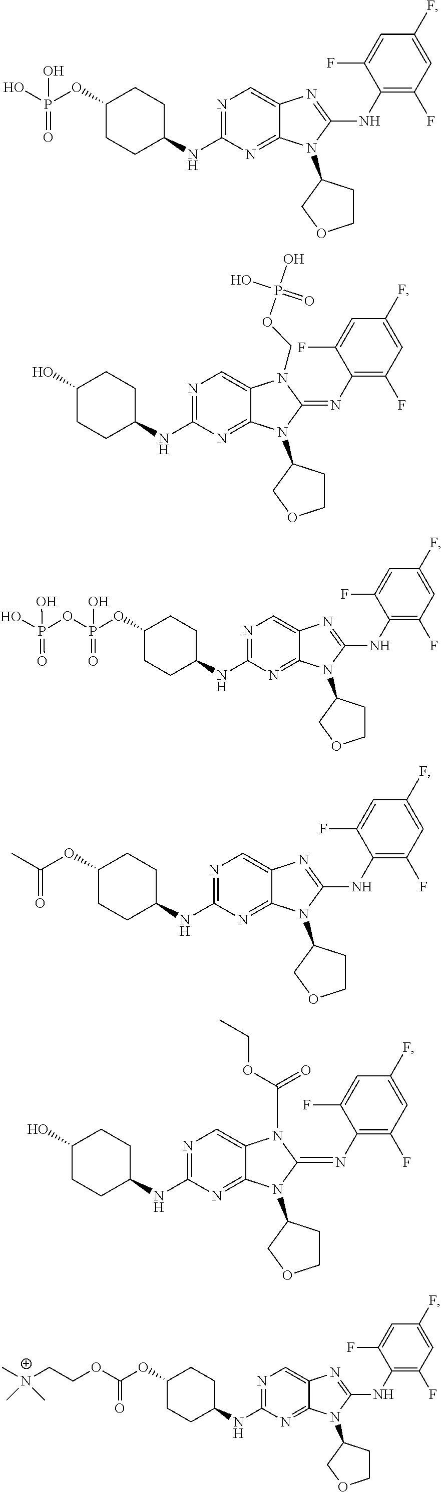

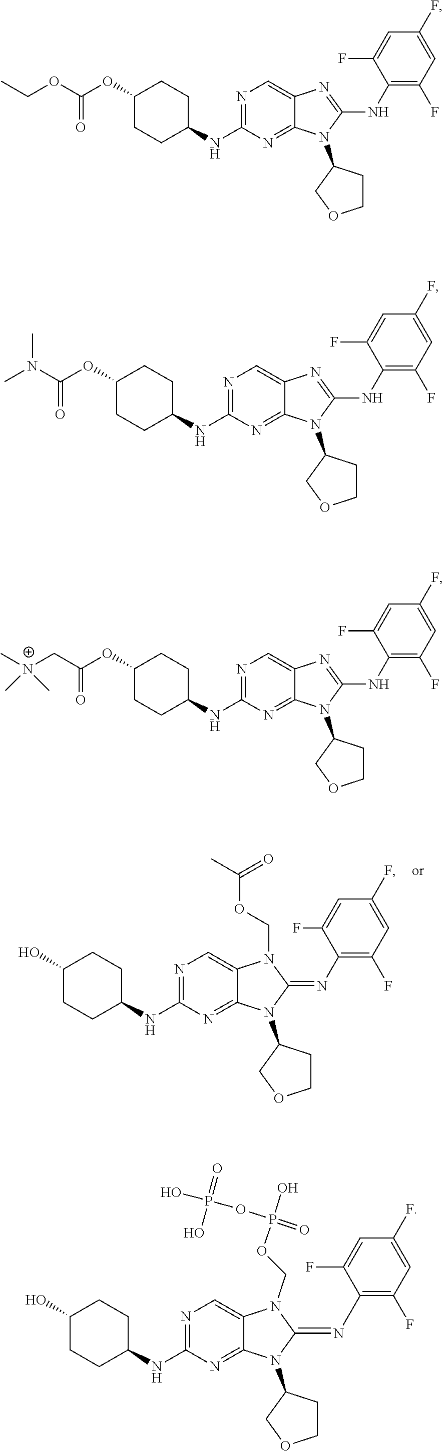 [SMILES: CCOC(=O)O[C@H]1CC[C@H](Nc2ncc3nc(Nc4c(F)cc(F)cc4F)n([C@H]4CCOC4)c3n2)CC1 ; CN(C)C(=O)O[C@H]1CC[C@H](Nc2ncc3nc(Nc4c(F)cc(F)cc4F)n([C@H]4CCOC4)c3n2)CC1 ; C[N+](C)(C)CC(=O)O[C@H]1CC[C@H](Nc2ncc3nc(Nc4c(F)cc(F)cc4F)n([C@H]4CCOC4)c3n2)CC1 ; CC(=O)OCn1/c(=N\c2c(F)cc(F)cc2F)n([C@H]2CCOC2)c2nc(N[C@H]3CC[C@H](O)CC3)ncc21 ; O=P(O)(O)OP(=O)(O)OCn1/c(=N\c2c(F)cc(F)cc2F)n([C@H]2CCOC2)c2nc(N[C@H]3CC[C@H](O)CC3)ncc21]

In an exemplary embodiment, the compound, or a salt or a tautomer or an isomer thereof, has a structure which is

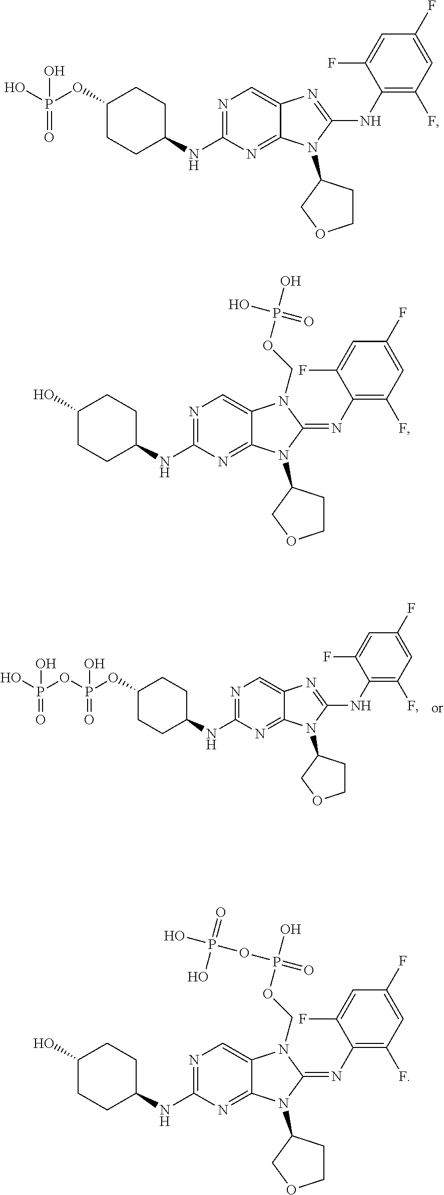 [SMILES: O=P(O)(O)O[C@H]1CC[C@H](Nc2ncc3nc(Nc4c(F)cc(F)cc4F)n([C@H]4CCOC4)c3n2)CC1 ; O=P(O)(O)OCn1/c(=N\c2c(F)cc(F)cc2F)n([C@H]2CCOC2)c2nc(N[C@H]3CC[C@H](O)CC3)ncc21 ; O=P(O)(O)OP(=O)(O)O[C@H]1CC[C@H](Nc2ncc3nc(Nc4c(F)cc(F)cc4F)n([C@H]4CCOC4)c3n2)CC1 ; O=P(O)(O)OP(=O)(O)OCn1/c(=N\c2c(F)cc(F)cc2F)n([C@H]2CCOC2)c2nc(N[C@H]3CC[C@H](O)CC3)ncc21]

In an exemplary embodiment, the compound, or a salt or a tautomer or an isomer thereof, wherein the salt is a pharmaceutically acceptable salt.

In one aspect, the invention is a salt of a compound of the invention. In an exemplary embodiment, the invention is a salt of a compound described herein. In an exemplary embodiment, the invention is a salt of a compound according to a formula described herein.

In one aspect, the invention is a pharmaceutically acceptable salt of a compound of the invention. In an exemplary embodiment, the invention is a pharmaceutically acceptable salt of a compound described herein. In an exemplary embodiment, the invention is a pharmaceutically acceptable salt of a compound according to a formula described herein.

In an exemplary embodiment, the compound, or a salt or a tautomer or an isomer thereof, wherein the salt of the compound is aluminum, arginine, benzathine, calcium, chloroprocaine, choline, diethanolamine, ethylenediamine, histidine, lithium, lysine, magnesium, meglumine, potassium, procaine, sodium, triethylamine, or zinc, or a combination thereof. In an exemplary embodiment, the compound, or a salt or a tautomer or an isomer thereof, wherein the pharmaceutically acceptable salt of the compound is aluminum, arginine, benzathine, calcium, chloroprocaine, choline, diethanolamine, ethylenediamine, histidine, lithium, lysine, magnesium, meglumine, potassium, procaine, sodium, triethylamine, or zinc, or a combination thereof.

In an exemplary embodiment, the compound, or a salt or a tautomer or an isomer thereof, has a structure which is

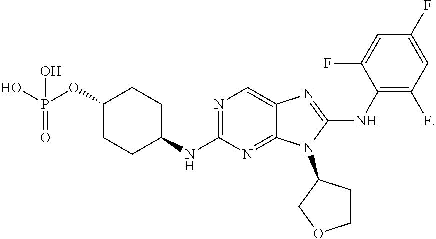 [SMILES: O=P(O)(O)O[C@H]1CC[C@H](Nc2ncc3nc(Nc4c(F)cc(F)cc4F)n([C@H]4CCOC4)c3n2)CC1]

In an exemplary embodiment, the invention is a salt of

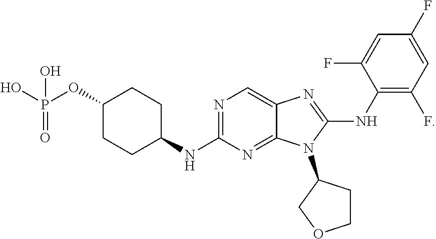 [SMILES: O=P(O)(O)O[C@H]1CC[C@H](Nc2ncc3nc(Nc4c(F)cc(F)cc4F)n([C@H]4CCOC4)c3n2)CC1]

In an exemplary embodiment, the invention is a pharmaceutically acceptable salt of

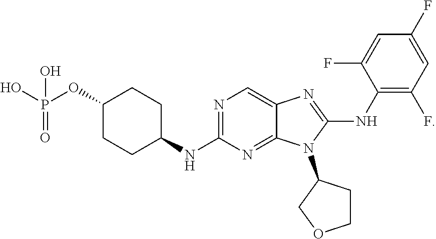 [SMILES: O=P(O)(O)O[C@H]1CC[C@H](Nc2ncc3nc(Nc4c(F)cc(F)cc4F)n([C@H]4CCOC4)c3n2)CC1]

In an exemplary embodiment, the invention is a pharmaceutically acceptable salt of

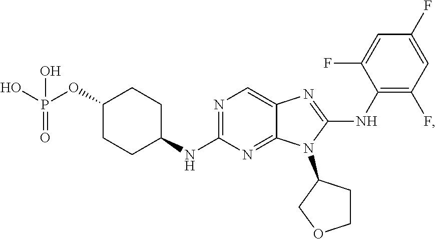 [SMILES: O=P(O)(O)O[C@H]1CC[C@H](Nc2ncc3nc(Nc4c(F)cc(F)cc4F)n([C@H]4CCOC4)c3n2)CC1]

wherein the pharmaceutically acceptable salt is aluminum, arginine, benzathine, calcium, chloroprocaine, choline, diethanolamine, ethylenediamine, histidine, lithium, lysine, magnesium, meglumine, potassium, procaine, sodium, triethylamine, or zinc, or a combination thereof. In an exemplary embodiment, the invention is a pharmaceutically acceptable salt of

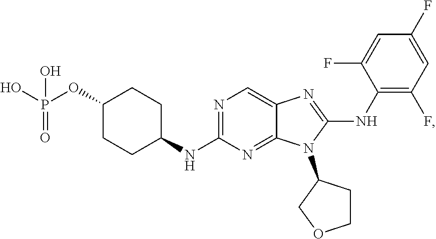 [SMILES: O=P(O)(O)O[C@H]1CC[C@H](Nc2ncc3nc(Nc4c(F)cc(F)cc4F)n([C@H]4CCOC4)c3n2)CC1]

wherein the pharmaceutically acceptable salt is sodium. In an exemplary embodiment, the invention is a pharmaceutically acceptable salt of

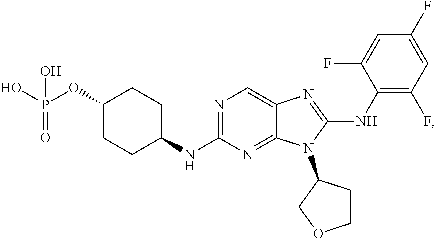 [SMILES: O=P(O)(O)O[C@H]1CC[C@H](Nc2ncc3nc(Nc4c(F)cc(F)cc4F)n([C@H]4CCOC4)c3n2)CC1]

wherein the pharmaceutically acceptable salt is a monosodium salt. In an exemplary embodiment, the invention is a pharmaceutically acceptable salt of

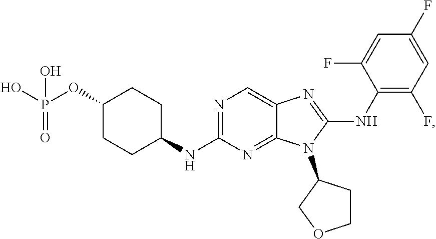 [SMILES: O=P(O)(O)O[C@H]1CC[C@H](Nc2ncc3nc(Nc4c(F)cc(F)cc4F)n([C@H]4CCOC4)c3n2)CC1]

wherein the pharmaceutically acceptable salt is a disodium salt.

In an exemplary embodiment, the compound, or a salt or a tautomer or an isomer thereof, has a structure which is

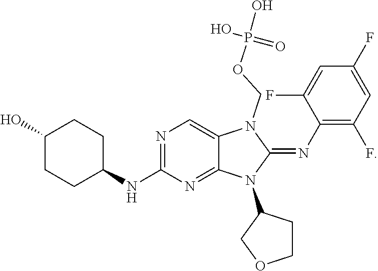

In an exemplary embodiment, the invention is a salt of

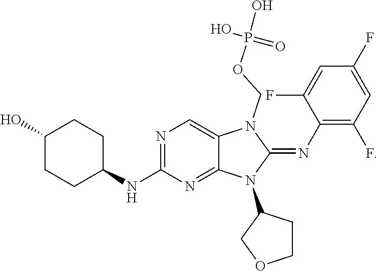

In an exemplary embodiment, the invention is a pharmaceutically acceptable salt of

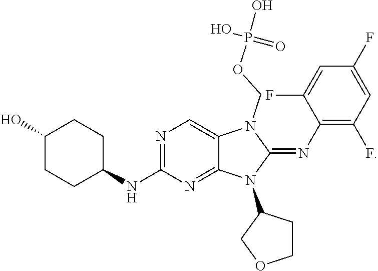

In an exemplary embodiment, the invention is a pharmaceutically acceptable salt of

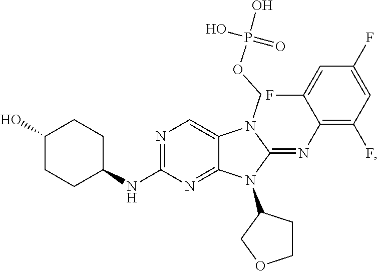

wherein the pharmaceutically acceptable salt is aluminum, arginine, benzathine, calcium, chloroprocaine, choline, diethanolamine, ethylenediamine, histidine, lithium, lysine, magnesium, meglumine, potassium, procaine, sodium, triethylamine, or zinc, or a combination thereof. In an exemplary embodiment, the invention is a pharmaceutically acceptable salt of

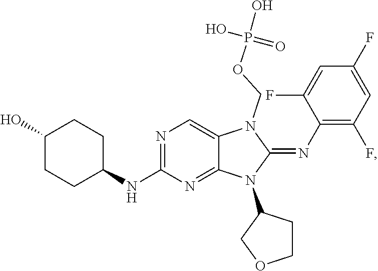

wherein the pharmaceutically acceptable salt is sodium. In an exemplary embodiment, the invention is a pharmaceutically acceptable salt of

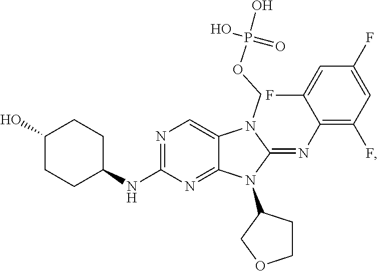

wherein the pharmaceutically acceptable salt is a monosodium salt. In an exemplary embodiment, the invention is a pharmaceutically acceptable salt of

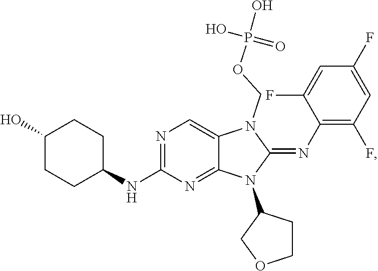

wherein the pharmaceutically acceptable salt is a disodium salt.

In an exemplary embodiment, the compound, or a salt or a tautomer or an isomer thereof, has a structure which is

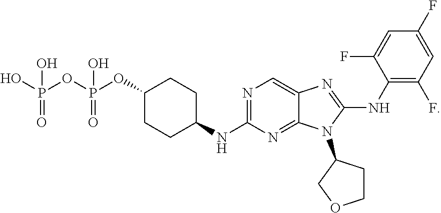 [SMILES: O=P(O)(O)OP(=O)(O)O[C@H]1CC[C@H](Nc2ncc3nc(Nc4c(F)cc(F)cc4F)n([C@H]4CCOC4)c3n2)CC1]

In an exemplary embodiment, the invention is a salt of

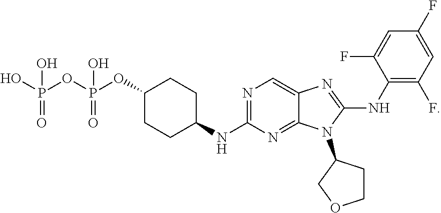 [SMILES: O=P(O)(O)OP(=O)(O)O[C@H]1CC[C@H](Nc2ncc3nc(Nc4c(F)cc(F)cc4F)n([C@H]4CCOC4)c3n2)CC1]

In an exemplary embodiment, the invention is a pharmaceutically acceptable salt of

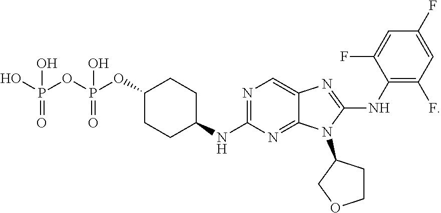 [SMILES: O=P(O)(O)OP(=O)(O)O[C@H]1CC[C@H](Nc2ncc3nc(Nc4c(F)cc(F)cc4F)n([C@H]4CCOC4)c3n2)CC1]

In an exemplary embodiment, the invention is a pharmaceutically acceptable salt of

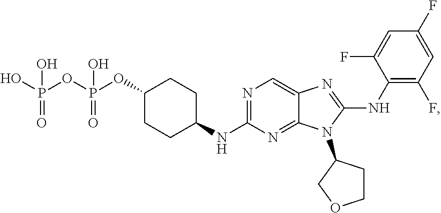 [SMILES: O=P(O)(O)OP(=O)(O)O[C@H]1CC[C@H](Nc2ncc3nc(Nc4c(F)cc(F)cc4F)n([C@H]4CCOC4)c3n2)CC1]

wherein the pharmaceutically acceptable salt is aluminum, arginine, benzathine, calcium, chloroprocaine, choline, diethanolamine, ethylenediamine, histidine, lithium, lysine, magnesium, meglumine, potassium, procaine, sodium, triethylamine, or zinc, or a combination thereof. In an exemplary embodiment, the invention is a pharmaceutically acceptable salt of

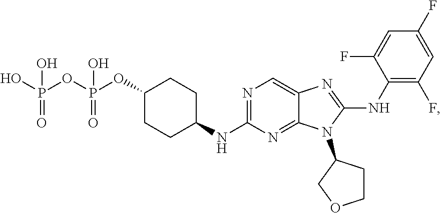 [SMILES: O=P(O)(O)OP(=O)(O)O[C@H]1CC[C@H](Nc2ncc3nc(Nc4c(F)cc(F)cc4F)n([C@H]4CCOC4)c3n2)CC1]

wherein the pharmaceutically acceptable salt is sodium. In an exemplary embodiment, the invention is a pharmaceutically acceptable salt of

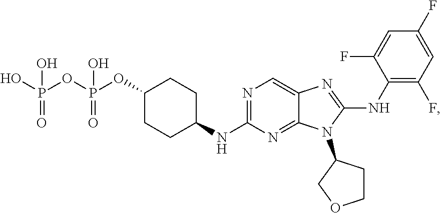 [SMILES: O=P(O)(O)OP(=O)(O)O[C@H]1CC[C@H](Nc2ncc3nc(Nc4c(F)cc(F)cc4F)n([C@H]4CCOC4)c3n2)CC1]

wherein the pharmaceutically acceptable salt is a monosodium salt. In an exemplary embodiment, the invention is a pharmaceutically acceptable salt of

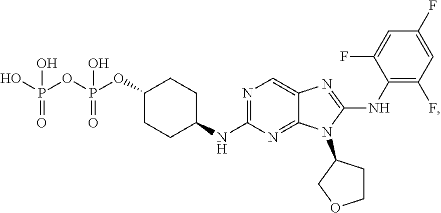 [SMILES: O=P(O)(O)OP(=O)(O)O[C@H]1CC[C@H](Nc2ncc3nc(Nc4c(F)cc(F)cc4F)n([C@H]4CCOC4)c3n2)CC1]

wherein the pharmaceutically acceptable salt is a disodium salt. In an exemplary embodiment, the invention is a pharmaceutically acceptable salt of

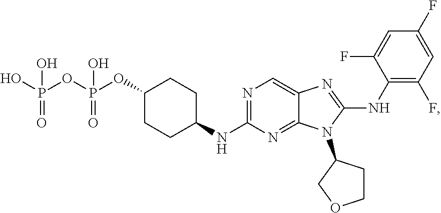 [SMILES: O=P(O)(O)OP(=O)(O)O[C@H]1CC[C@H](Nc2ncc3nc(Nc4c(F)cc(F)cc4F)n([C@H]4CCOC4)c3n2)CC1]

wherein the pharmaceutically acceptable salt is a trisodium salt.

In an exemplary embodiment, the compound, or a salt or a tautomer or an isomer thereof, has a structure which is

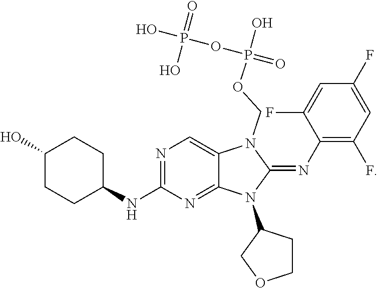

In an exemplary embodiment, the invention is a salt of

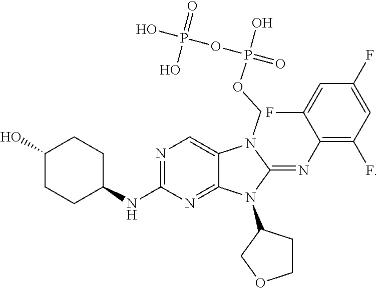

In an exemplary embodiment, the invention is a pharmaceutically acceptable salt of

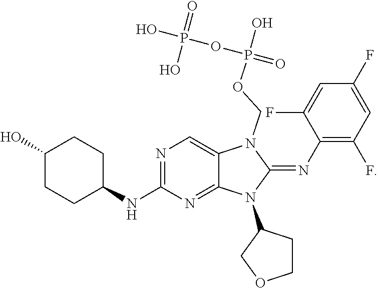

In an exemplary embodiment, the invention is a pharmaceutically acceptable salt of

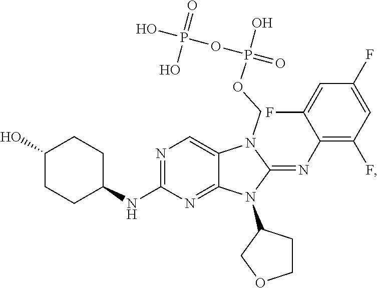

wherein the pharmaceutically acceptable salt is aluminum, arginine, benzathine, calcium, chloroprocaine, choline, diethanolamine, ethylenediamine, histidine, lithium, lysine, magnesium, meglumine, potassium, procaine, sodium, triethylamine, or zinc, or a combination thereof. In an exemplary embodiment, the invention is a pharmaceutically acceptable salt of

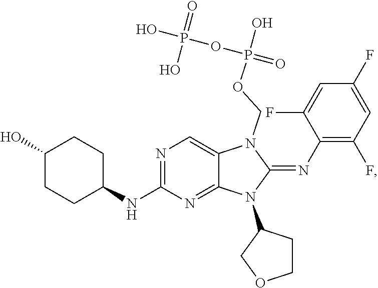

wherein the pharmaceutically acceptable salt is sodium. In an exemplary embodiment, the invention is a pharmaceutically acceptable salt of

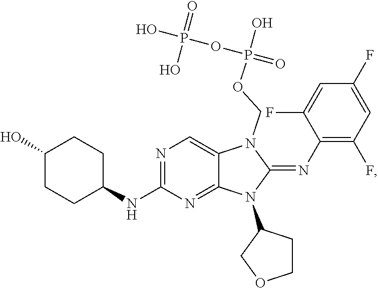

wherein the pharmaceutically acceptable salt is a monosodium salt. In an exemplary embodiment, the invention is a pharmaceutically acceptable salt of

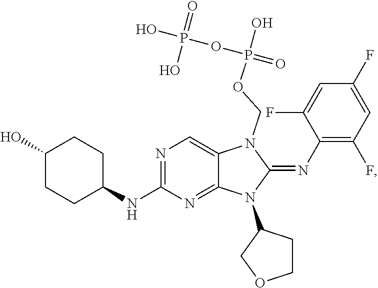

wherein the pharmaceutically acceptable salt is a disodium salt. In an exemplary embodiment, the invention is a pharmaceutically acceptable salt of

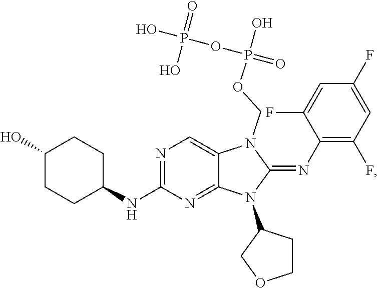

wherein the pharmaceutically acceptable salt is a trisodium salt.

In an exemplary embodiment, the compound, or a salt or a tautomer or an isomer thereof, which is a disodium salt of

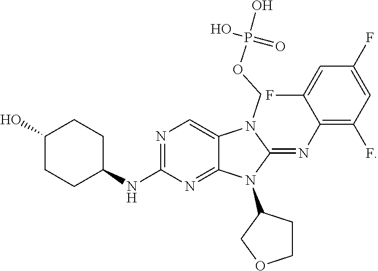

In an exemplary embodiment, the compound, or a salt or a tautomer or an isomer thereof, which is a trisodium salt of

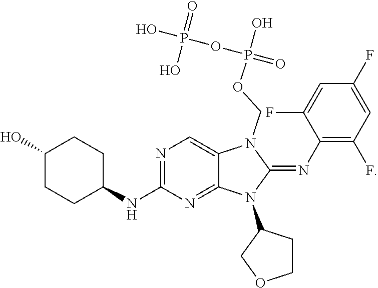

### IV. Methods

In an exemplary embodiment, the invention provides a method of treating and/or preventing an injury before, during, or after a surgical procedure to a subject, comprising: administering to the subject a therapeutically and/or prophylactically effective amount of a compound described herein, or a salt or a tautomer or an isomer thereof, or a pharmaceutical formulation described herein, thereby treating and/or preventing the injury before, during, or after the surgical procedure. In an exemplary embodiment, the surgical procedure is on an organ of the subject, and the organ is selected from the group consisting of kidney, lung, heart, brain, and liver. In an exemplary embodiment, the surgical procedure is on a kidney of the subject. In an exemplary embodiment, the surgical procedure is on a lung of the subject. In an exemplary embodiment, the surgical procedure is on a heart of the subject. In an exemplary embodiment, the surgical procedure is on a brain of the subject. In an exemplary embodiment, the surgical procedure is on a liver of the subject. In an exemplary embodiment, the surgical procedure is on a peripheral limb or spinal cord of the subject. In an exemplary embodiment, the injury is ischemia, ischemic reperfusion, embolism, thrombosis, sepsis, toxicity associated with chemotherapy, toxicity associated with radiotherapy, toxicity associated with a nephrotoxin, or associated with physical trauma. In an exemplary embodiment, the injury is associated with physical trauma, such as being subject to an explosion or being subject to a rapid deceleration event. In an exemplary embodiment, the subject is not otherwise in need of treatment with the compound described herein, or a salt or a tautomer or an isomer thereof. In an exemplary embodiment, the subject is not otherwise in need of treatment with

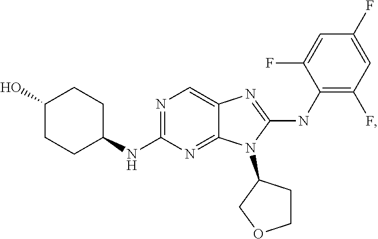 [SMILES: O[C@H]1CC[C@H](Nc2ncc3nc(Nc4c(F)cc(F)cc4F)n([C@H]4CCOC4)c3n2)CC1]

or a salt or a tautomer or an isomer thereof.

In an exemplary embodiment, the injury is to an organ of the subject, and the organ is selected from the group consisting of kidney, lung, heart, brain, and liver. In an exemplary embodiment, the injury is to a kidney of the subject. In an exemplary embodiment, the injury is to a lung of the subject. In an exemplary embodiment, the injury is to a heart of the subject. In an exemplary embodiment, the injury is to a brain of the subject. In an exemplary embodiment, the injury is to a liver of the subject. In an exemplary embodiment, the injury is to a peripheral limb or spinal cord of the subject. In an exemplary embodiment, the injury is ischemic reperfusion to an organ of the subject, and the organ is selected from the group consisting of kidney, lung, heart, brain, and liver. In an exemplary embodiment, the injury is ischemic reperfusion to a kidney of the subject. In an exemplary embodiment, the injury is ischemic reperfusion to a lung of the subject. In an exemplary embodiment, the injury is ischemic reperfusion to a heart of the subject. In an exemplary embodiment, the injury is ischemic reperfusion to a brain of the subject. In an exemplary embodiment, the injury is ischemic reperfusion to a liver of the subject. In an exemplary embodiment, the injury is ischemic reperfusion to a peripheral limb or spinal cord of the subject.

In an exemplary embodiment, the invention is a method of treating and/or preventing ischemic reperfusion injury to a kidney before, during, or after a surgical procedure on a subject, comprising: administering to the subject a therapeutically and/or prophylactically effective amount of a compound described herein, or a salt or a tautomer or an isomer thereof, or a pharmaceutical formulation described herein, thereby treating and/or preventing the ischemic reperfusion injury to the kidney before, during, or after the surgical procedure on the subject. In an exemplary embodiment, the invention is a method of treating and/or preventing ischemic reperfusion injury to a kidney before, during, or after a surgical procedure on a subject, comprising: administering to the subject a therapeutically and/or prophylactically effective amount of a compound having a structure which is:

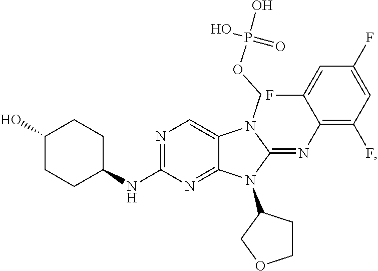

or a salt or a tautomer or an isomer thereof, thereby treating and/or preventing the ischemic reperfusion injury to the kidney before, during, or after the surgical procedure on the subject. In an exemplary embodiment, the subject is an animal. In an exemplary embodiment, the subject is a human. In an exemplary embodiment, the subject is not otherwise in need of treatment with the compound described in this paragraph, or a salt or a tautomer or an isomer thereof.

In an exemplary embodiment, the invention is a method of treating and/or preventing ischemic reperfusion injury before, during, or after a surgical procedure to a kidney of a subject, comprising: administering to the subject a therapeutically and/or prophylactically effective amount of a compound described herein, or a salt or a tautomer or an isomer thereof, or a pharmaceutical formulation described herein, thereby treating and/or preventing the ischemic reperfusion injury before, during, or after the surgical procedure to the kidney of the subject. In an exemplary embodiment, the invention is a method of treating and/or preventing ischemic reperfusion injury before, during, or after a surgical procedure to a kidney of a subject, comprising: administering to the subject a therapeutically and/or prophylactically effective amount of a compound having a structure which is:

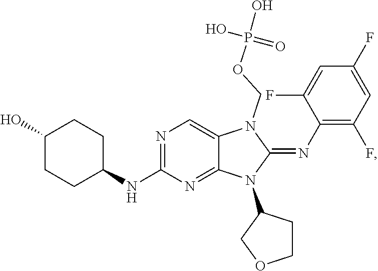

or a salt or a tautomer or an isomer thereof, thereby treating and/or preventing the ischemic reperfusion injury before, during, or after the surgical procedure to the kidney of the subject. In an exemplary embodiment, the subject is an animal. In an exemplary embodiment, the subject is a human. In an exemplary embodiment, the subject is not otherwise in need of treatment with the compound described in this paragraph, or a salt or a tautomer or an isomer thereof.

In an exemplary embodiment, the invention provides a method of treating and/or preventing an injury before, during, or after a surgical procedure to a subject, comprising: administering to the subject a therapeutically and/or prophylactically effective amount of a compound having a structure which is:

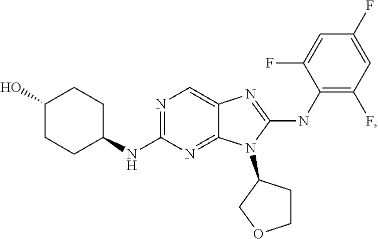 [SMILES: O[C@H]1CC[C@H](Nc2ncc3nc(Nc4c(F)cc(F)cc4F)n([C@H]4CCOC4)c3n2)CC1]

or a salt or a tautomer or an isomer thereof, or a pharmaceutical formulation described herein, thereby treating and/or preventing the injury before, during, or after the surgical procedure. In an exemplary embodiment, the surgical procedure is on an organ of the subject, and the organ is selected from the group consisting of kidney, lung, heart, brain, and liver. In an exemplary embodiment, the surgical procedure is on a kidney of the subject. In an exemplary embodiment, the surgical procedure is on a lung of the subject. In an exemplary embodiment, the surgical procedure is on a heart of the subject. In an exemplary embodiment, the surgical procedure is on a brain of the subject. In an exemplary embodiment, the surgical procedure is on a liver of the subject. In an exemplary embodiment, the surgical procedure is on a peripheral limb or spinal cord of the subject. In an exemplary embodiment, the injury is ischemia, ischemic reperfusion, embolism, thrombosis, sepsis, toxicity associated with chemotherapy, toxicity associated with radiotherapy, toxicity associated with a nephrotoxin, or associated with physical trauma. In an exemplary embodiment, the injury is associated with physical trauma, such as being subject to an explosion or being subject to a rapid deceleration event. In an exemplary embodiment, the injury is to an organ of the subject, and the organ is selected from the group consisting of kidney, lung, heart, brain, and liver. In an exemplary embodiment, the injury is to a kidney of the subject. In an exemplary embodiment, the injury is to a lung of the subject. In an exemplary embodiment, the injury is to a heart of the subject. In an exemplary embodiment, the injury is to a brain of the subject. In an exemplary embodiment, the injury is to a liver of the subject. In an exemplary embodiment, the injury is to a peripheral limb or spinal cord of the subject. In an exemplary embodiment, the injury is ischemic reperfusion to an organ of the subject, and the organ is selected from the group consisting of kidney, lung, heart, brain, and liver. In an exemplary embodiment, the injury is ischemic reperfusion to a kidney of the subject. In an exemplary embodiment, the injury is ischemic reperfusion to a lung of the subject. In an exemplary embodiment, the injury is ischemic reperfusion to a heart of the subject. In an exemplary embodiment, the injury is ischemic reperfusion to a brain of the subject. In an exemplary embodiment, the injury is ischemic reperfusion to a liver of the subject. In an exemplary embodiment, the injury is ischemic reperfusion to a peripheral limb or spinal cord of the subject. In an exemplary embodiment, the subject is not otherwise in need of treatment with the compound described in this paragraph, or a salt or a tautomer or an isomer thereof.

In an exemplary embodiment, the invention is a method of treating and/or preventing ischemic reperfusion injury to a kidney before, during, or after a surgical procedure on a subject, comprising: administering to the subject a therapeutically and/or prophylactically effective amount of a compound having a structure which is:

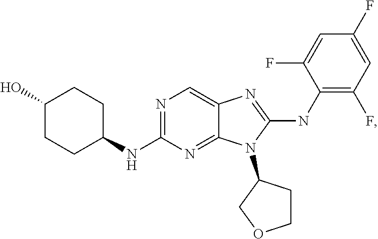 [SMILES: O[C@H]1CC[C@H](Nc2ncc3nc(Nc4c(F)cc(F)cc4F)n([C@H]4CCOC4)c3n2)CC1]

or a salt or a tautomer or an isomer thereof, or a pharmaceutical formulation described herein, thereby treating and/or preventing the ischemic reperfusion injury to the kidney before, during, or after the surgical procedure on the subject. In an exemplary embodiment, the invention is a method of treating and/or preventing ischemic reperfusion injury to a kidney before, during, or after a surgical procedure on a subject, comprising: administering to the subject a therapeutically and/or prophylactically effective amount of a compound having a structure which is:

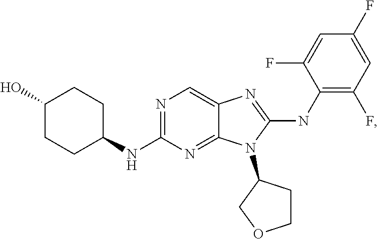 [SMILES: O[C@H]1CC[C@H](Nc2ncc3nc(Nc4c(F)cc(F)cc4F)n([C@H]4CCOC4)c3n2)CC1]

or a salt or a tautomer or an isomer thereof, thereby treating and/or preventing the ischemic reperfusion injury to the kidney before, during, or after the surgical procedure on the subject. In an exemplary embodiment, the subject is an animal. In an exemplary embodiment, the subject is a human. In an exemplary embodiment, the invention is a method of treating and/or preventing ischemic reperfusion injury before, during, or after a surgical procedure to a kidney of a subject, comprising: administering to the subject a therapeutically and/or prophylactically effective amount of a compound described herein, or a salt or a tautomer or an isomer thereof, or a pharmaceutical formulation described herein, thereby treating and/or preventing the ischemic reperfusion injury before, during, or after the surgical procedure to the kidney of the subject. In an exemplary embodiment, the invention is a method of treating and/or preventing ischemic reperfusion injury before, during, or after a surgical procedure to a kidney of a subject, comprising: administering to the subject a therapeutically and/or prophylactically effective amount of a compound having a structure which is:

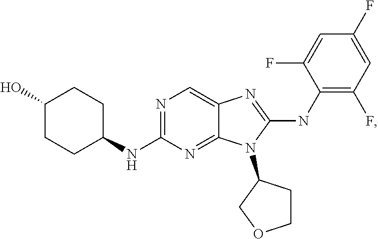 [SMILES: O[C@H]1CC[C@H](Nc2ncc3nc(Nc4c(F)cc(F)cc4F)n([C@H]4CCOC4)c3n2)CC1]

or a salt or a tautomer or an isomer thereof, thereby treating and/or preventing the ischemic reperfusion injury before, during, or after the surgical procedure to the kidney of the subject. In an exemplary embodiment, the subject is an animal. In an exemplary embodiment, the subject is a human. In an exemplary embodiment, the subject is not otherwise in need of treatment with the compound described in this paragraph, or a salt or a tautomer or an isomer thereof. In an exemplary embodiment, the invention provides a method of treating and/or preventing an injury before, during, or after a surgical procedure to a subject, comprising: administering to the subject a therapeutically and/or prophylactically effective amount of a compound having a structure which is:

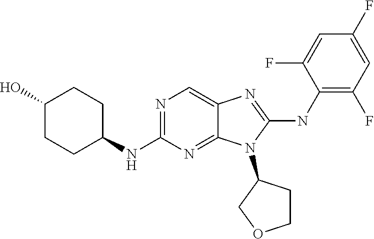 [SMILES: O[C@H]1CC[C@H](Nc2ncc3nc(Nc4c(F)cc(F)cc4F)n([C@H]4CCOC4)c3n2)CC1]

or a salt or a tautomer or an isomer thereof, and wherein the subject is not otherwise in need of treatment and/or prevention with the compound or a salt or a tautomer or an isomer thereof, thereby treating and/or preventing the injury before, during, or after the surgical procedure to the subject. In an exemplary embodiment, the invention provides a method of treating and/or preventing ischemic reperfusion injury before, during, or after a surgical procedure to a kidney of a subject, comprising: administering to the subject a therapeutically and/or prophylactically effective amount of a compound having a structure which is:

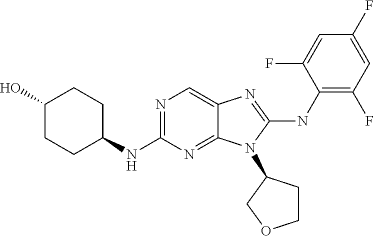 [SMILES: O[C@H]1CC[C@H](Nc2ncc3nc(Nc4c(F)cc(F)cc4F)n([C@H]4CCOC4)c3n2)CC1]

or a salt or a tautomer or an isomer thereof, and wherein the subject is not otherwise in need of treatment and/or prevention with the compound or a salt or a tautomer or an isomer thereof, thereby treating and/or preventing the ischemic reperfusion injury before, during, or after the surgical procedure to the kidney of the subject.

In an exemplary embodiment, the invention provides a method of inhibiting a c-Jun N-terminal kinase (JNK) in a mammal, comprising administering an effective amount of a compound of the invention to a mammal, wherein the mammal is not otherwise is need of JNK inhibition. In an exemplary embodiment, the mammal is a human.

### V. Pharmaceutical Formulations

In another aspect, the invention is a pharmaceutical formulation which includes: (a) a pharmaceutically acceptable excipient; and (b) a compound of the invention. In another aspect, the pharmaceutical formulation includes: (a) a pharmaceutically acceptable excipient; and (b) a compound according to a formula described herein. In another aspect, the pharmaceutical formulation includes: (a) a pharmaceutically acceptable excipient; and (b) a compound described herein, or a pharmaceutically acceptable salt or a tautomer or an isomer thereof, or a combination thereof. In another aspect, the pharmaceutical formulation includes: (a) a pharmaceutically acceptable excipient; and (b) a compound described herein, or a pharmaceutically acceptable salt or a tautomer or an isomer thereof, or a combination thereof. In another aspect, the pharmaceutical formulation includes: (a) a pharmaceutically acceptable excipient; and (b) a compound described herein, or a pharmaceutically acceptable salt or a tautomer or an isomer thereof. In another aspect, the pharmaceutical formulation includes: (a) a pharmaceutically acceptable excipient; and (b) a pharmaceutically acceptable salt of a compound described herein. In another exemplary embodiment, the pharmaceutical formulation includes: (a) a pharmaceutically acceptable excipient; and (b) a compound described herein. In an exemplary embodiment, the pharmaceutical formulation is a unit dosage form. In an exemplary embodiment, the pharmaceutical formulation is a single unit dosage form.

The pharmaceutical formulations of the invention can take a variety of forms adapted to the chosen route of administration. In an exemplary embodiment, the pharmaceutical formulation is an intravenous formulation. In an exemplary embodiment, the pharmaceutical formulation is an intravenous formulation, and comprises saline. In an exemplary embodiment, the pharmaceutical formulation is an intravenous formulation, and comprises phosphate buffered saline.

The invention is further illustrated by the Examples that follow. The Examples are not intended to define or limit the scope of the invention.

### EXAMPLES

The following Examples illustrate the synthesis of representative compounds used in the invention and the following Reference Examples illustrate the synthesis of intermediates in their preparation. These examples are not intended, nor are they to be construed, as limiting the scope of the invention. It will be clear that the invention may be practiced otherwise than as particularly described herein. Numerous modifications and variations of the invention are possible in view of the teachings herein and, therefore, are within the scope of the invention.

In the Examples below, unless otherwise indicated, all temperatures are set forth in degrees Celsius and all parts and percentages are by weight. Reagents may be purchased from commercial suppliers, such as Sigma-Aldrich Chemical Company, and may be used without further purification unless otherwise indicated. Reagents may also be prepared following standard literature procedures known to those skilled in the art. Solvents may be purchased from Sigma-Aldrich in Sure-Seal bottles and used as received. All solvents may be purified using standard methods known to those skilled in the art, unless otherwise indicated. The reactions set forth below were run generally at ambient temperature, unless otherwise indicated. The reaction vessels were fitted with rubber septa for introduction of substrates and reagents via syringe. Analytical thin layer chromatography (TLC) was performed using glass-backed silica gel pre-coated plates (Amalec TLC Uniplates™ with fluorescent indicator) and eluted with appropriate solvent ratios (v/v). Reactions were assayed by TLC or LC/MS, and terminated as judged by the consumption of starting material. Visualization of the TLC plates was done with UV light (254 nm wavelength) or with an appropriate TLC visualizing solvent, such as basic aqueous KMnO₄ solution, ninhydrin, cerium molybdate, or phosphomolybdic acid, activated with heat. Flash column chromatography (W. C. Still et al., J. Org. Chem., 43, 1978, 2923-2925) was performed using Biotage Isolera Prime automated flash purification system (220 and 254 nm wavelength) with ZIP Sphere-spherical Silica or KP Silica cartridges or various preparative HPLC systems. The compound structures in the examples below were confirmed by one or more of the following methods: proton magnetic resonance spectroscopy, mass spectrometry, and melting point. Proton magnetic resonance (¹HNMR) spectra were recorded using an NMR spectrometers operating at 300, 400 or 500 MHz field strength. Chemical shifts are reported in the form of delta (6) values given in parts per million (ppm) relative to an internal standard, such as tetramethylsilane (TMS). Alternatively, ¹HNMR spectra were referenced to signals from residual protons in deuterated solvents as follows: CDCl₃=7.25 ppm; DMSO-d₆=2.49 ppm; CD₃OD=3.30 ppm. Peak multiplicities are designated as follows: s, singlet; d, doublet; dd, doublet of doublets; t, triplet; dt, doublet of triplets; q, quartet; br, broadened; and m, multiplet. Coupling constants are given in Hertz (Hz). Mass spectrometric (MS) data were obtained using a mass spectrometer with APCI or ESI ionization.

Starting materials used were either available from commercial sources or prepared according to literature procedures and had experimental data in accordance with those reported.

### Example 1

### Compounds of the Invention

Compounds of the invention have the following structures and exemplary syntheses:

### Q7: [4-[[9-[(3S)-Tetrahydro furan-3-yl]-8-(2,4,6-trifluoroanilino)purin-2-yl]amino]cyclohexyl] disodium phosphate

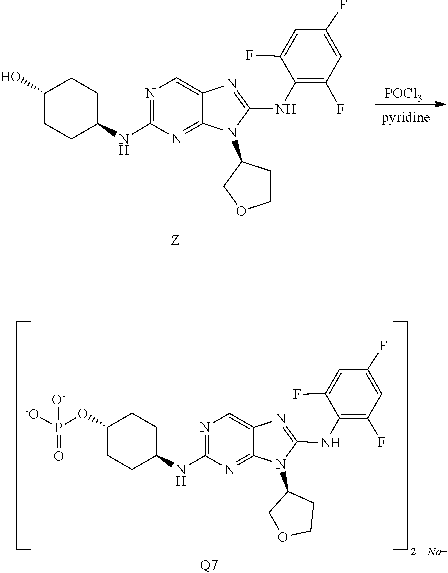

To a solution of compound Z (300 mg, 0.67 mmol, 1.0 eq.) in anhydrous THF (6 mL) at 0° C. was added pyridine (2-3 drops), followed by POCl₃ (205 mg, 1.34 mmol, 2.0 eq.) dropwise. The mixture was stirred at 0° C. for 1 h and rt for another 1 h. The mixture was monitored by LC-MS. The mixture was concentrated under reduced pressure. The residue was dissolved in methanol and treated with saturated sodium bicarbonate to pH 8. The mixture was purified with reverse phase chromatography (C18 column, acetonitrile and water) to give Q7 (100 mg, 26%) as white solid.

LCMS: m/z: 529.30 [M+1]⁺; ³¹P NMR(162 MHz, D₂O) δ 3.08; ¹H NMR (400 MHz, CD₃OD) δ 7.81 (s, 1H), 7.02 (t, J=8.4 Hz, 2H), 5.35 (s, 1H), 4.42-4.29 (m, 2H), 4.23-4.14 (m, 1H), 4.09 (t, J=8.3 Hz, 1H), 3.98-3.93 (m, 1H), 3.85-3.78 (m, 1H), 2.62-2.58 (m, 2H), 2.25-2.06 (m, 4H), 1.68-1.40 (m, 4H).

### Q8: [[(8Z)-2-[(4-hydroxycyclohexyl)aminol-9-[(3S)-tetrahydrofuran-3-yl]-8-(2,4,6-trifluorophenyl)imino-purin-7-yl]methoxy-sodiooxy-phosphoryl]oxysodium

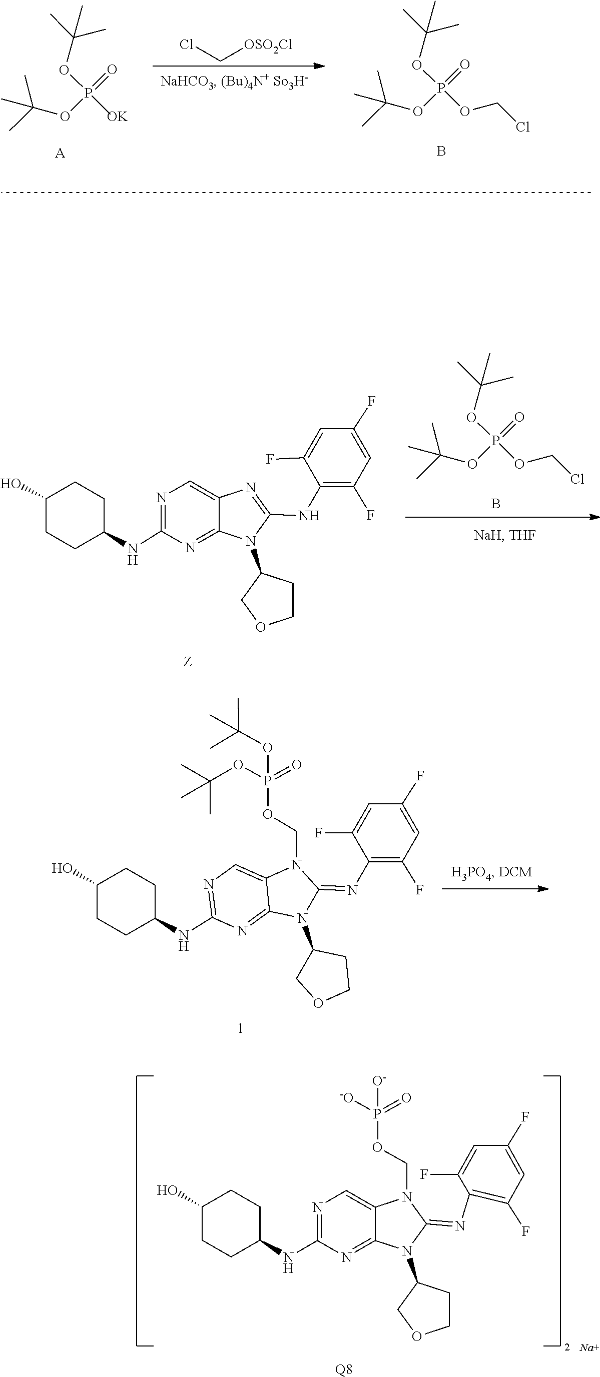

### Step 1: Ditert-butyl chloromethyl phosphate

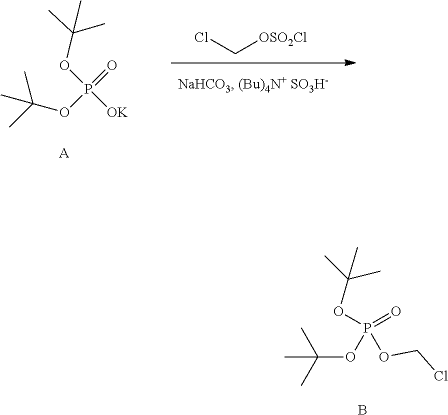

To a solution of ditert-butoxyphosphoryloxypotassium (2 g, 8.05 mmol, 1 eq) in DCM (10 mL) and H₂O (10 mL) was added NaHCO₃ (2.71 g, 32.22 mmol, 1.25 mL, 4 eq) and hydrogen sulfate;tetrabutylammonium (273.49 mg, 805.49 umol, 0.1 eq) at 0° C. Then chloro(chlorosulfonyloxy)methane (1.99 g, 12.08 mmol, 1.5 eq) was added dropwise to the reaction. The mixture was stirred at 15° C. for 12 h. TLC (PE:EA=3:1) showed the reaction was complete and one new spot (R_{f}=0.43) was detected. Water (10 mL) was charged to fully dissolve solids, the layers were split, and the lower product-rich DCM layer was washed with 5 wt % aqueous potassium carbonate (12 mL). The layers were then split, and the lower product-rich DCM layer was charged solid K₂CO₃ (12 mg) and concentrated under reduced pressure to give ditert-butyl chloromethyl phosphate (1.7 g, 6.57 mmol, 81.59% yield) as light yellow oil. (contained K₂CO₃ solid).

### Step 2: Ditert-butyl [(8Z)-2-[(4-hydroxycyclohexyl)amino]-9-[(3S)-tetrahydrofuran-3-yl]-8-(2,4,6-trifluorophenyl)imino-purin-7-yl]methyl phosphate

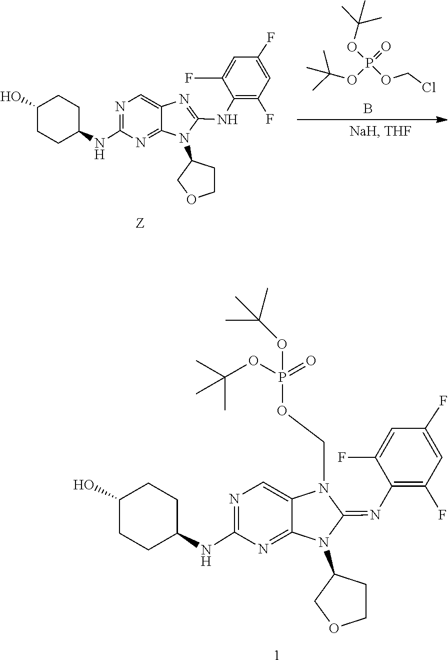

To a mixture of Z (1 g, 2.23 mmol, 1 eq) in THF (20 mL) was added NaH (445.95 mg, 11.15 mmol, 60% purity, 5 eq) at 0° C., after stirred at 0° C. for 0.5 h, ditert-butyl chloromethyl phosphate (1.15 g, 4.46 mmol, 2 eq) was added to the above mixture, then the mixture was stirred at 30° C. for 12 h. Additional 1.15 g of Cpd.B was added to the reaction mixture, then the mixture was stirred at 30° C. for another 12 h. The reaction mixture was quenched by water 20 mL and then extracted with EtOAc 30 mL (10 mL*3). The combined organic layers were dried over anhydrous Na₂SO₄, filtered and concentrated under reduced pressure to give a residue. The crude product was purified by column chromatography (SiO₂, Petroleum ether/Ethyl acetate=1/1 to 0:1) to give ditert-butyl [(8Z)-2-[(4-hydroxycyclohexyl) amino]-9-[(3S)-tetrahydrofuran-3-yl]-8-(2,4,6-trifluorophenyl)imino-purin-7-yl]methyl phosphate (860 mg, 1.28 mmol, 57.50% yield) as yellow oil.

LC-MS: 671.4 [M+1]⁺

### Step 3: [[(8Z)-2-[(4-hydroxycyclohexyl)amino]-9-[(3S)-tetrahydrofuran-3-yl]-8-(2,4,6-trifluorophenyl)imino-purin-7-yl]methoxy-sodiooxy-phosphoryl]oxysodium

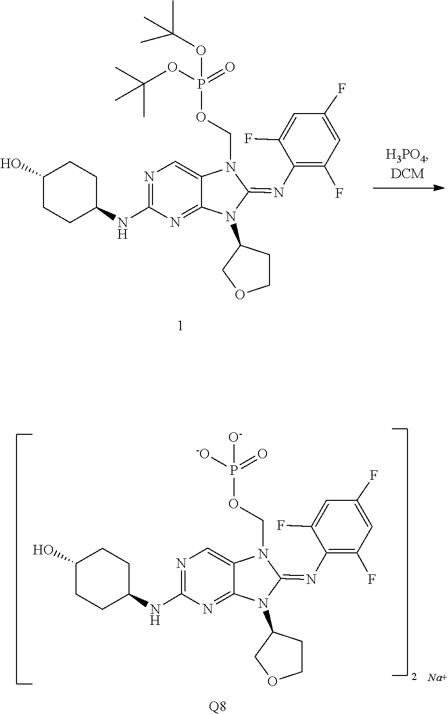

To a mixture of ditert-butyl [(8Z)-2-[(4-hydroxycyclohexyl)amino]-9-[(3S)-tetrahydrofuran-3-yl]-8-(2,4,6-trifluorophenyl)imino-purin-7-yl]methyl phosphate (0.86 g, 1.28 mmol, 1 eq) in DCM (15 mL) was added H₃PO₄ (628.34 mg, 6.41 mmol, 374.01 uL, 5 eq), then the mixture was stirred at 20° C. for 0.5 h. LCMS showed Cpd.1 was consumed and desired mass was detected. The reaction mixture was concentrated to remove DCM. The crude product was dissolved with MeOH (3 mL), then aq. 5 NaHCO₃ was added to the mixture to adjust pH to 8, the mixture washed with EtOAc (10 mL*2). The aqueous phase was concentrated to give the crude product. The crude product was purified by prep-HPLC (column: Welch Ultimate AQ-C18 150*30 mm*5 um;mobile phase: [Water-ACN];B %: 2%-30%,20 min) to give [[(8Z)-2-[(4-hydroxycyclohexyl)amino]-9-[(3S)-tetrahydrofuran-3-yl]-8-(2,4,6-trifluorophenyl)imino-purin-7-yl]methoxy-sodiooxy-phosphoryl]oxysodium (Q8) (200 mg, 320.11 umol, 24.96% yield, 96.420% purity) as a white solid.

LCMS: 559.2 [M+1]⁺; ³¹P NMR (162 MHz, DEUTERIUM OXIDE) 6 ppm 1.54 (s, 1 P); ¹H NMR (400 MHz, DEUTERIUM OXIDE) 6 ppm 1.02-1.53 (m, 4H) 1.81-2.09 (m, 4H) 2.15-2.30 (m, 1H) 2.51 (td, J=12.88, 5.63 Hz, 1H) 3.44-3.69 (m, 2H) 3.80-4.00 (m, 2H) 4.09-4.31 (m, 2H) 4.89-5.04 (m, 1H) 5.16 (d, J=5.63 Hz, 2H) 6.85 (br t, J=8.88 Hz, 2H) 7.94 (s, 1H)

### Q9: [Disodiooxyphosphoryloxy-[4-[[9-[(3S)-tetrahydrofuran-3-yl]-8-(2,4,6-trifluoroanilino)purin-2-yl]amino]cyclohexoxy]phosphoryl]oxysodium

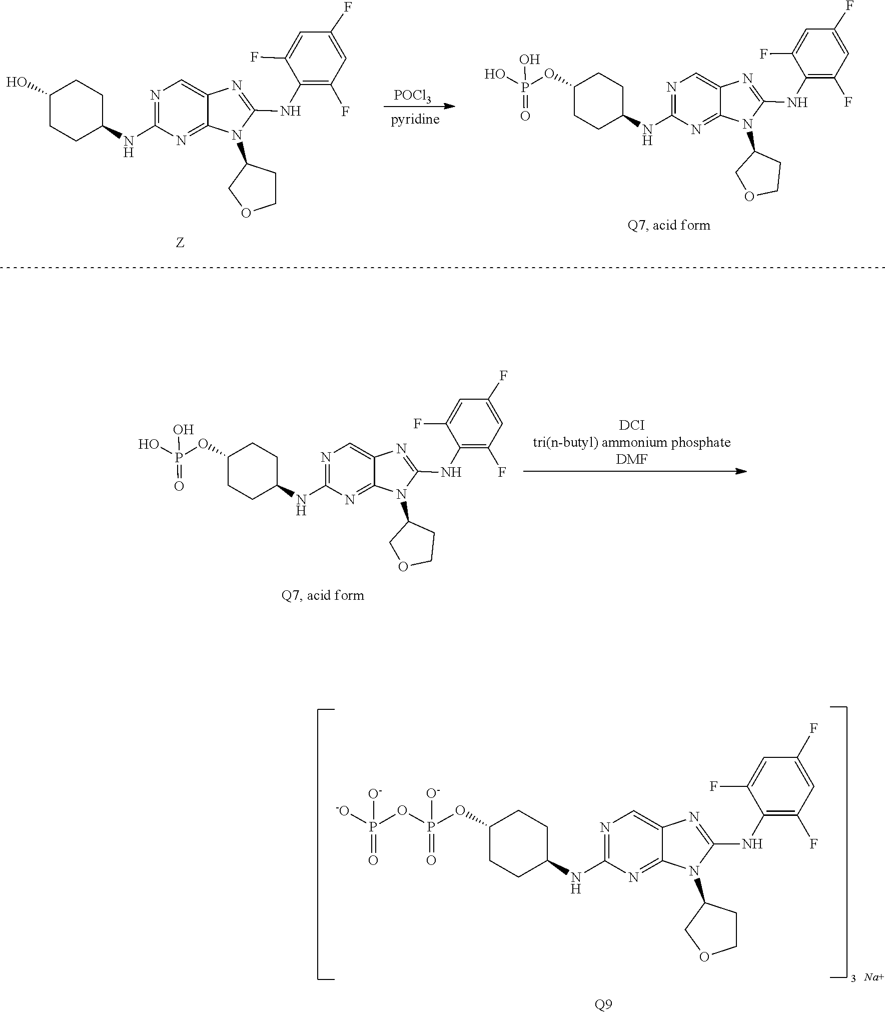

### STEP 1: [4-[[9-[(3S)-tetrahydrofuran-3-yl]-8-(2,4,6-trifluoroanilino)purin-2-yl]amino]cyclohexyl]dihydrogen phosphate (Q7, acid form)

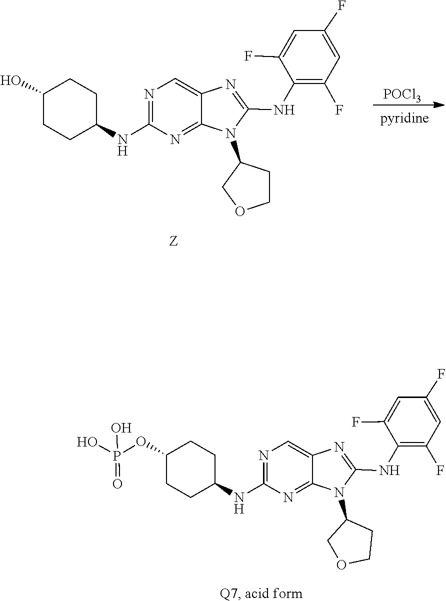

To a solution of 4-[[9-[(3S)-tetrahydrofuran-3-yl]-8-(2,4,6-trifluoroanilino)purin-2-yl]amino]cyclohexanol (“Z”) (500 mg, 1.11 mmol, 1 eq) and pyridine (264.58 mg, 3.34 mmol, 269.98 uL, 3 eq) in PO(OMe)₃ (10 mL) was added POCl₃ (341.92 mg, 2.23 mmol, 207.23 uL, 2 eq) at −10° C., then the mixture was stirred at −10° C. for 0.5 h. LCMS showed Z was almost consumed and the desired mass was detected. The reaction mixture was quenched by 1M TetraEthylAmmonium Bromide (“TEAB”) (6 mL). The mixture was filtered and the filtrate was purified by prep-HPLC (neutral condition; column: Waters Xbridge C18 150*50 mm*10 um;mobile phase: [water(10 mM NH₄HCO₃)-ACN];B %: 1%-40%,10 min) to give [4-[[9-[(3S)-tetrahydrofuran-3-yl]-8-(2,4,6-trifluoroanilino)purin-2-yl]amino]cyclohexyl]dihydrogen phosphate (Q7, acid form) (320 mg, 605.58 umol, 54.31% yield) as a white solid.

LC-MS: 527.1 [M+1]⁺

### STEP 2: [Disodiooxyphosphoryloxy-[4-[[9-[(3S)-tetrahydrofuran-3-yl]-8-(2,4,6-trifluoroanilino)purin-2-yl]amino]cyclohexoxy]phosphoryl]oxysodium

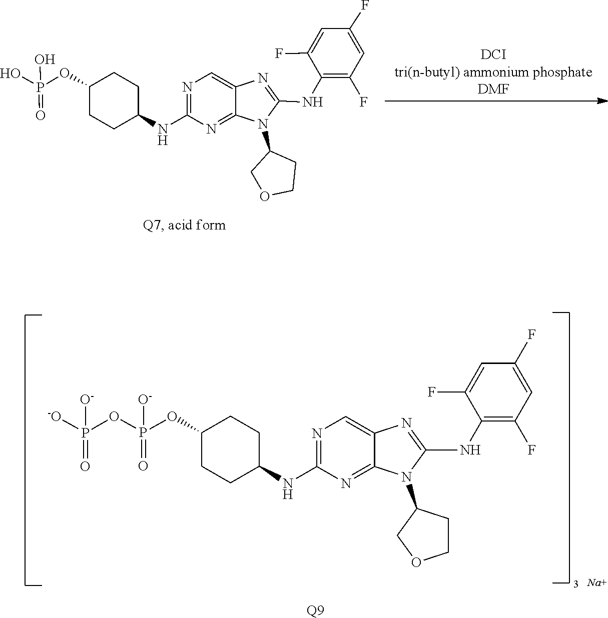

To a solution of Q7 (acid form, 200 mg, 378.49 umol, 1 eq) in DMF (2 mL)was added CDI (306.86 mg, 1.89 mmol, 5 eq), the mixture was stirred at 20° C. for 2 h. LCMS showed the reaction was complete and desired intermediate was detected. Excess of CDI was eliminated by addition of MeOH (97.02 mg, 3.03 mmol, 122.53 uL, 8 eq) and the mixture was stirred for 30 min. Then dibutyl-phosphonooxy-propyl-ammonium (1 M, 3.78 mL, 10 eq) were added. The mixture was stirred for another 12 h. LCMS showed the reaction was completely and the desired mass was detected. The mixture was filtered and the filtrate was concentrated to give the crude product. The mixture was filtered and the filtrate was purified by prep-HPLC (neutral condition; column: Waters Xbridge C18 150*50 mm*10 um; mobile phase: [water(10 mM NH₄HCO₃)-ACN]; B %: 1%-30%, 10 min), then the product was exchanged to sodium salt through sodium ion exchange resin. [disodiooxyphosphoryloxy-[4-[[9-[(3S)-tetrahydrofuran-3-yl]-8-(2,4,6-trifluoroanilino)purin-2-yl]amino]cyclohexoxy]phosphoryl]oxysodium (Q9) (145 mg, 210.65 umol, 55.65% yield, 97.964% purity) was obtained as a white solid.

LC-MS: 609.0 [M+1]⁺; ¹H NMR (400 MHz, DEUTERIUM OXIDE) 6 ppm 1.30-1.44 (m, 2H) 1.46-1.64 (m, 2H) 1.97-2.22 (m, 4H) 2.42-2.63 (m, 2H) 3.68 (tt, J=10.79, 3.72 Hz, 1H) 3.95 (q, J=7.96 Hz, 1H) 4.05 (dd, J=10.01, 7.00 Hz, 1H) 4.14-4.24 (m, 1H) 4.26-4.42 (m, 2H) 5.18-5.38 (m, 1H) 6.96 (t, J=8.38 Hz, 2H) 7.86 (s, 1H); ³¹P NMR (162 MHz, DEUTERIUM OXIDE) δ ppm-11.93-−11.43 (m, 1 P) −10.48 (d, J=19.62 Hz, 1 P); ¹⁹F NMR (377 MHz, DEUTERIUM OXIDE) δ ppm-119.31-−115.10 (m, 1 F) −109.95-−106.91 (m, 1 F).

### Q10: [4-[[9-[(3S)-Tetrahydrofuran-3-yl]-8-(2,4,6-trifluoroanilino)purin-2-yl]amino]cyclohexyl] acetate

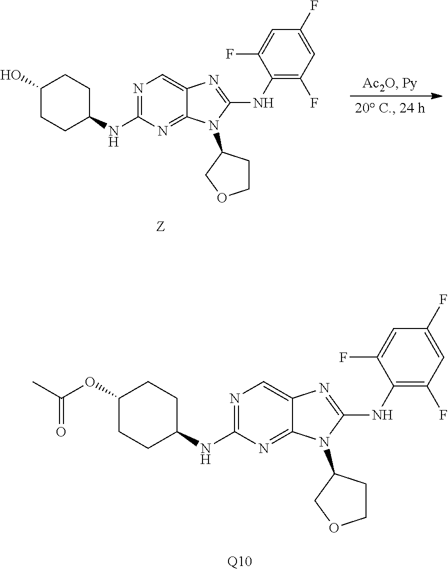 [SMILES: O[C@H]1CC[C@H](Nc2ncc3nc(Nc4c(F)cc(F)cc4F)n([C@H]4CCOC4)c3n2)CC1 ; "20° C., 24 h" ; "Ac2O, Py" ; CC(=O)O[C@H]1CC[C@H](Nc2ncc3nc(Nc4c(F)cc(F)cc4F)n([C@H]4CCOC4)c3n2)CC1 ; "Q10"]

### Step 1: [4-[[9-[(3S)-tetrahydrofuran-3-yl]-8-(2,4,6-trifluoroanilino)purin-2-yl]amino]cyclohexyl]acetate

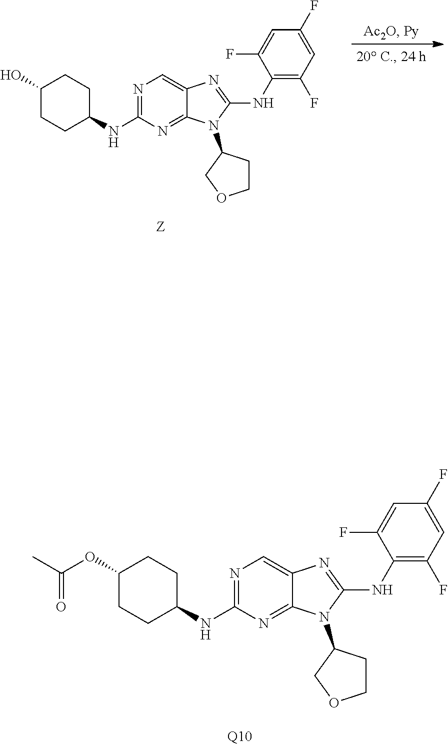 [SMILES: O[C@H]1CC[C@H](Nc2ncc3nc(Nc4c(F)cc(F)cc4F)n([C@H]4CCOC4)c3n2)CC1 ; "20° C., 24 h" ; "Ac2O, Py" ; CC(=O)O[C@H]1CC[C@H](Nc2ncc3nc(Nc4c(F)cc(F)cc4F)n([C@H]4CCOC4)c3n2)CC1 ; "Q10"]

To a solution of 4-[[9-[(3S)-tetrahydrofuran-3-yl]-8-(2,4,6-trifluoroanilino) purin-2-yl]amino]cyclohexanol (300 mg, 668.98 umol, 1 eq) in Py (5 mL) was added Ac₂O (136.59 mg, 1.34 mmol, 125.31 uL, 2 eq). The mixture was stirred at 20° C. for 12 h. LCMS showed Z was remained, the mixture was stirred at 20° C. for another 12 h. LCMS showed Z was almost consumed and desired mass was detected. The mixture was filtered and the filtrate was purified by prep-HPLC (neutral condition; column: Waters Xbridge BEH C18 100*30 mm*10 um; mobile phase: [water(10 mM NH₄HCO₃)-ACN]; B %: 30%-55%, 10 min) to give [4-[[9-[(3S)-tetrahydrofuran-3-yl]-8-(2,4,6-trifluoroanilino)purin-2-yl]amino]cyclohexyl]acetate (Q10) (130 mg, 264.49 umol, 39.54% yield, 99.788% purity) as a light yellow solid.

LC-MS: 491.2 [M+1]⁺; ¹H NMR (400 MHz, CHLOROFORM-d) δ ppm 1.26-1.42 (m, 2H) 1.50-1.63 (m, 2H) 2.02 (br s, 1H) 2.05 (s, 3H) 2.10-2.24 (m, 3H) 2.25-2.34 (m, 1H) 2.60-2.80 (m, 1H) 3.64-4.05 (m, 3H) 4.34-4.55 (m, 2H) 4.66-4.89 (m, 2H) 5.53 (br s, 1H) 6.79 (br t, J=8.07 Hz, 2H) 7.92 (s, 1H) 8.26 (s, 1H).

### Q11: Ethyl (8Z)-2-[(4-hydroxycyclohexyl)aminol-9-[(3S)-tetrahydrofuran-3-yl]-8-(2,4,6-trifluorophenyl)imino-purine-7-carboxylate

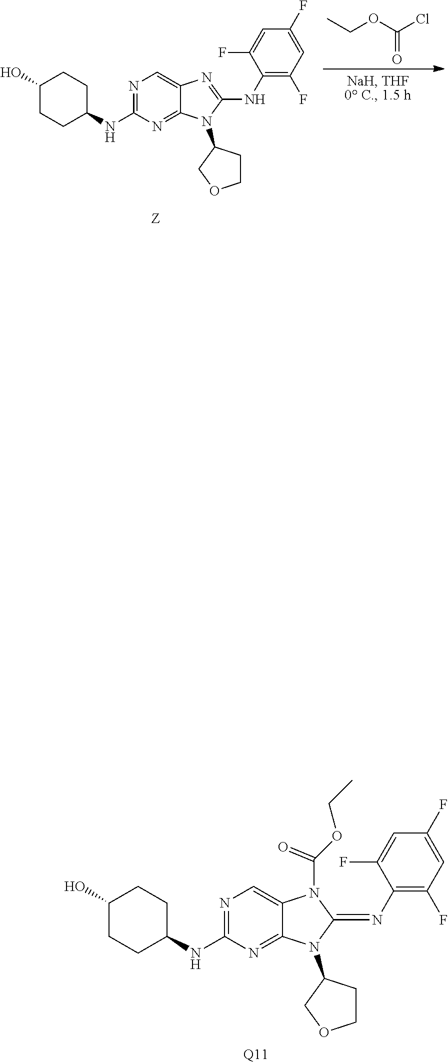

### Step 1: Ethyl (8Z)-2-[(4-hydroxycyclohexyl)amino]-9-[(3S)-tetrahydrofuran-3-yl]-8-(2,4,6-trifluorophenyl)imino-purine-7-carboxylate

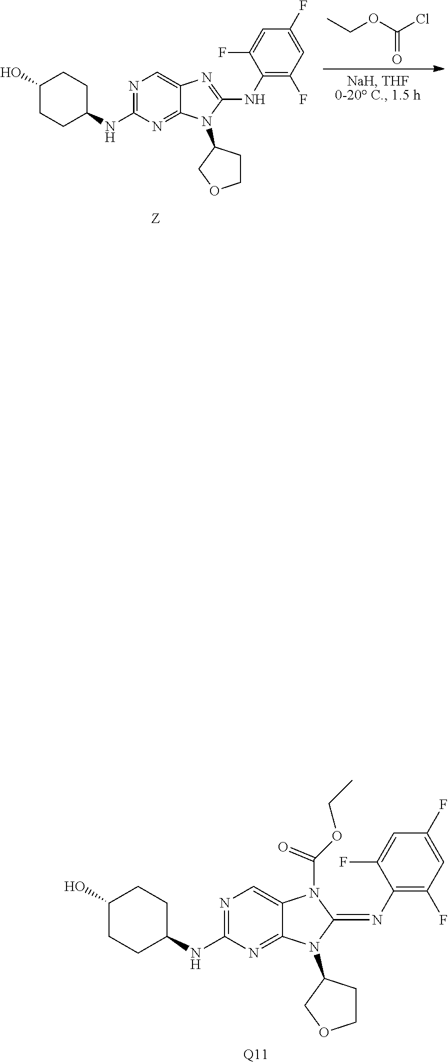

To a solution of 4-[[9-[(3S)-tetrahydrofuran-3-yl]-8-(2,4,6-trifluoroanilino) purin-2-yl]amino]cyclohexanol (0.2 g, 445.99 umol, 1 eq) in THF (3 mL) was added NaH (53.52 mg, 1.34 mmol, 46.56 uL, 60% purity, 3 eq) at 0° C., the mixture was stirred at 0° C. for 0.5 h, then ethyl carbonochloridate (72.60 mg, 668.98 umol, 63.68 uL, 1.5 eq) was added to the above mixture. The mixture was stirred at 20° C. for 1 h. LCMS showed Z was remained and desired mass was detected. The reaction mixture 10 was quenched by addition aq. NH₄Cl 4 mL, and extracted with DCM (3 mL*3). The combined organic layers were dried over Na₂SO₄, filtered and concentrated under reduced pressure to give a residue. The mixture was purified by prep-HPLC (neutral condition; column: Waters Xbridge Prep OBD C18 150*40 mm*10 um; mobile phase: [water(10 mM NH₄HCO₃)-ACN];B %: 30%-60%,8 min) to give ethyl (8Z)-2-[(4-hydroxycyclohexyl)amino]-9-[(3S)-tetrahydrofuran-3-yl]-8-(2,4,6-trifluorophenyl)imino-purine-7-carboxylate (59 mg, 111.93 umol, 25.10% yield, 98.743% purity) as a light yellow solid.

LC-MS: 521.2 [M+1]⁺; ¹H NMR (400 MHz, CHLOROFORM-d) δ ppm 1.19 (t, J=7.13 Hz, 3H) 1.25-1.35 (m, 2H) 1.40-1.47 (m, 2H) 1.99-2.10 (m, 2H) 2.17 (ddd, J=9.38, 6.57, 3.19 Hz, 2H) 2.25-2.37 (m, 1H) 2.71 (ddt, J=12.63, 7.54, 5.24, 5.24 Hz, 1H) 3.62-3.85 (m, 2H) 4.03 (td, J=7.97, 4.82 Hz, 1H) 4.08-4.31 (m, 6H) 4.81-5.13 (m, 1H) 5.23-5.54 (m, 1H) 6.58-6.80 (m, 2H) 8.26 (s, 1H); ¹⁹F NMR (377 MHz, CHLOROFORM-d) δ ppm-120.09 (br d, J=56.87 Hz, 1 F) −117.99 (br s, 1 F).

### Q12: Trimethyl-[2-[4-[[9-[(3S)-tetrahydrofuran-3-yl]-8-(2,4,6-trifluoroanilino)purin-2-yl]amino]cyclohexoxy]carbonyl]oxoethyl]ammonium chloride

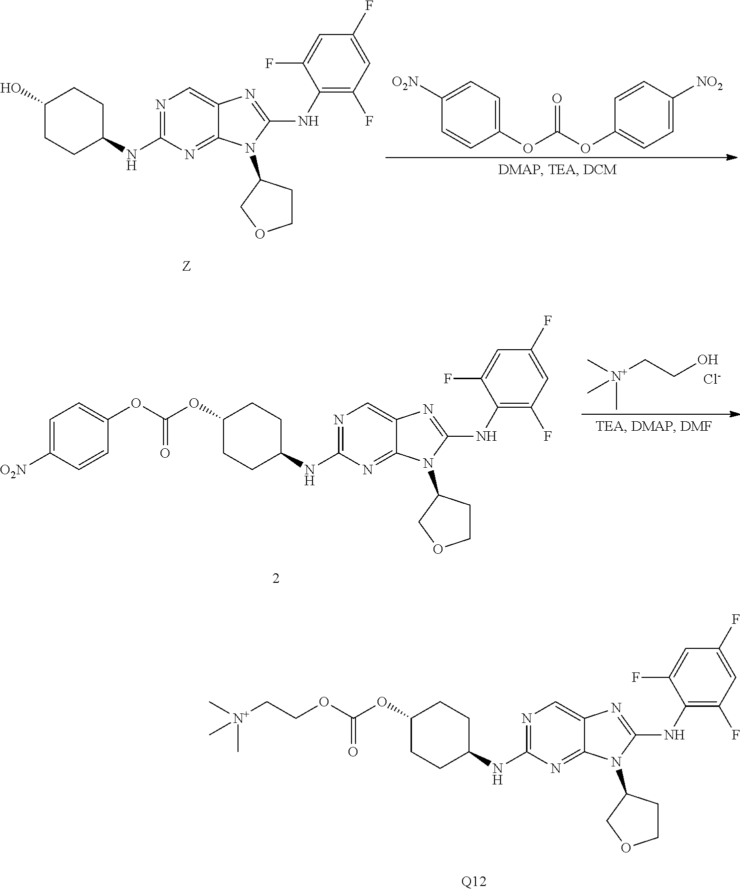

### Step 1: (4-nitrophenyl) [4-[[9-[(3S)-tetrahydrofuran-3-yl]-8-(2,4,6-trifluoroanilino)purin-2-yl]amino]cyclohexyl]carbonate

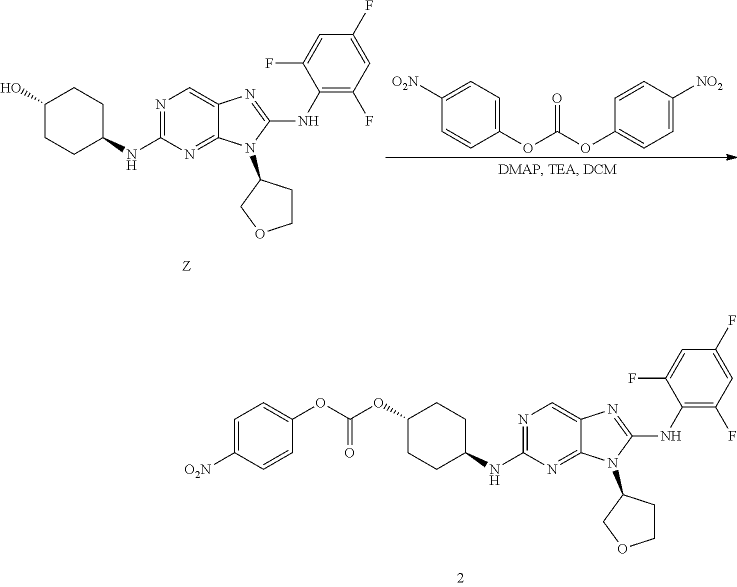

To a solution of 4-[[9-[(3S)-tetrahydrofuran-3-yl]-8-(2,4,6-trifluoroanilino)purin-2-yl]amino]cyclohexanol (1 g, 2.23 mmol, 1 eq) in DCM (20 mL) was added bis(4-nitrophenyl) carbonate (814.05 mg, 2.68 mmol, 1.2 eq) and DMAP (27.24 mg, 222.99 umol, 0.1 eq), TEA (225.65 mg, 2.23 mmol, 310.38 uL, 1 eq), then the mixture was stirred at 20° C. for 12 h. LCMS showed Z was almost consumed and the desired mass was detected. The mixture was concentrated to get a crude product. The residue was purified by prep-TLC (SiO₂, petroleum ether:ethyl acetate=0:1, Rt=0.64) to give (4-nitrophenyl) [4-[[9-[(3S)-tetrahydrofuran-3-yl]-8-(2,4,6-trifluoroanilino)purin-2-yl]amino]cyclohexyl]carbonate (1.2 g, 1.96 mmol, 87.71% yield) as a yellow solid.

LC-MS: 614.3 [M+1]⁺

### Step 2: Trimethyl-[2-[4-[[9-[(3S)-tetrahydrofuran-3-yl]-8-(2,4,6-trifluoroanilino) purin-2-yl]amino]cyclohexoxy]carbonyloxyethyl]ammonium chloride

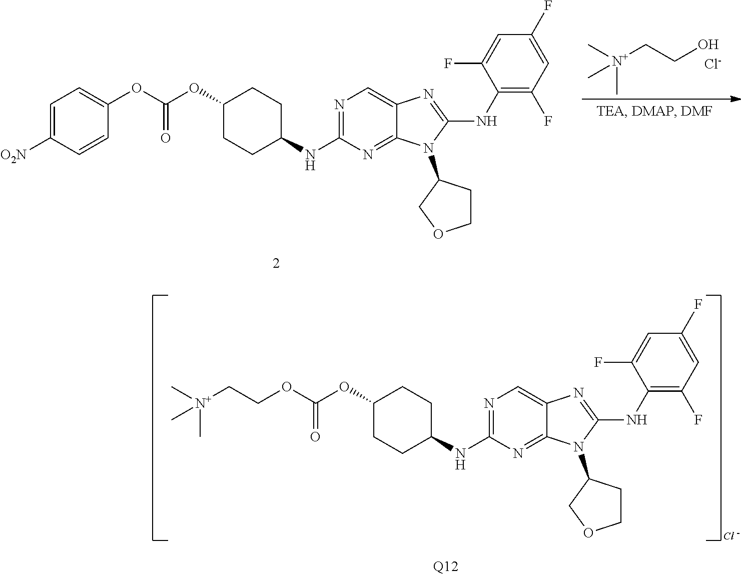

A mixture of (4-nitrophenyl) [4-[[9-[(3S)-tetrahydrofuran-3-yl]-8-(2,4,6-trifluoroanilino)purin-2-yl]amino]cyclohexyl]carbonate (350 mg, 570.46 umol, 1 eq), 2-hydroxyethyl(trimethyl)ammonium;chloride (398.24 mg, 2.85 mmol, 5 eq), DMAP (6.97 mg, 57.05 umol, 0.1 eq), TEA (41.23 mg, 407.45 umol, 56.71 uL, 5 eq) in DMF (5 mL) was degassed and purged with N₂ for 3 times, and then the mixture was stirred at 80° C. for 12 h under N₂ atmosphere. LCMS showed the reaction was complete and desired mass was detected. The mixture was concentrated to get a crude product. The crude product was purified by prep-HPLC (FA condition; column: Phenomenex Luna C18 200*40 mm*10 um;mobile phase: [water(0.2% FA)-ACN];B %: 10%-55%,8 min) to give trimethyl-[2-[4-[[9-[(3S)-tetrahydrofuran-3-yl]-8-(2,4,6-trifluoroanilino)purin-2-yl]amino]cyclohexoxy]carbonyloxyethyl]ammonium (Q12) (130 mg, 208.12 umol, 36.48% yield, 100% purity, FA) as a white solid.

LC-MS: 578.2 [M]⁺; ¹⁹F NMR (377 MHz, CHLOROFORM-d) δ ppm-117.19-114.88 (m, 1 F) −111.19-−107.50 (m, 1 F); ¹H NMR (400 MHz, CHLOROFORM-d) 6 ppm 1.24-1.46 (m, 2H) 1.51-1.73 (m, 2H) 2.02-2.44 (m, 5H) 2.52-2.79 (m, 1H) 3.44 (s, 9H) 3.71-4.01 (m, 3H) 4.12 (br s, 2H) 4.39 (br d, J=10.76 Hz, 1H) 4.46 (td, J=9.10, 3.44 Hz, 1H) 4.58-4.70 (m, 3H) 4.78 (br d, J=7.75 Hz, 1H) 5.40-5.64 (m, 1H) 6.65-6.92 (m, 2H) 8.24 (s, 1H) 8.67 (s, 1H).

### Q13: Ethyl [4-[[9-[(3S)-tetrahydrofuran-3-yl]-8-(2,4,6-trifluoroanilino)purin-2-yl]amino]cyclohexyl] carbonate

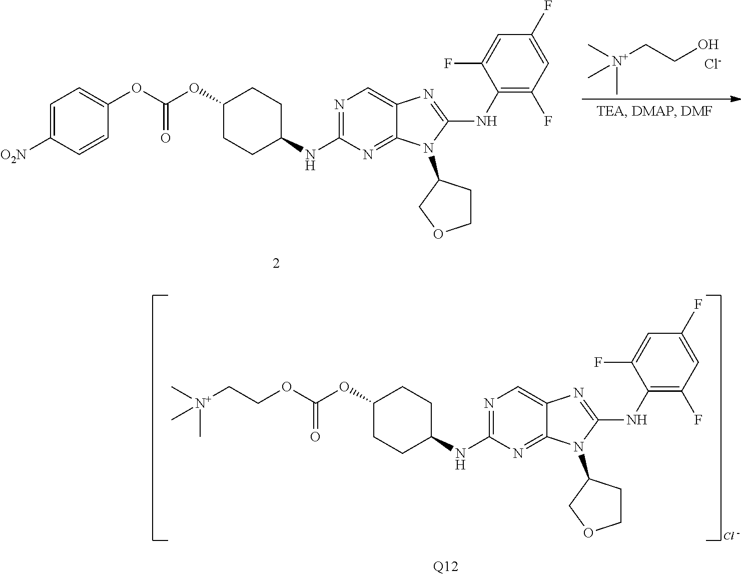

A mixture of (4-nitrophenyl) [4-[[9-[(3S)-tetrahydrofuran-3-yl]-8-(2,4,6-trifluoroanilino)purin-2-yl]amino]cyclohexyl]carbonate (250 mg, 407.47 umol, 1 eq), EtOH (375.43 mg, 8.15 mmol, 20 eq), DMAP (4.98 mg, 40.75 umol, 0.1 eq) TEA (412.32 mg, 4.07 mmol, 567.15 uL, 10 eq) in DCM (3 mL) was degassed and purged with N₂ for 3 times, and then the mixture was stirred at 40° C. for 12 h under N₂ atmosphere. LCMS showed the reaction was complete and desired mass was detected. The mixture was concentrated to get a crude product. The residue was purified by prep-HPLC (neutral condition, column: Waters Xbridge Prep OBD C18 150*40 mm*10 um; mobile phase: [water(10 mM NH₄HCO₃)-ACN];B %: 35%-55%,8 min) to give ethyl [4-t 9-[(3S)-tetrahydrofuran-3-yl]-8-(2,4,6-trifluoroanilino)purin-2-yl]amino]cyclohexyl]carbonate (Q13) (72.5 mg, 139.29 umol, 34.18% yield, 100% purity) as a white solid.

LC-MS: 521.2 [M+1]⁺; ¹H NMR (400 MHz, CHLOROFORM-d) δ ppm 1.30-1.42 (m, 5H) 1.58-1.70 (m, 2H) 2.09-2.34 (m, 5H) 2.62-2.80 (m, 1H) 3.71-3.99 (m, 3H) 4.09-4.29 (m, 2H) 4.36-4.53 (m, 2H) 4.54-4.71 (m, 1H) 4.76-4.89 (m, 1H) 5.55 (ddd, J=9.16, 5.97, 3.13 Hz, 1H) 6.80 (t, J=8.00 Hz, 2H) 7.93 (s, 1H) 8.26 (s, 1H); ¹⁹F NMR (377 MHz, CHLOROFORM-d) δ ppm-115.71 (d, J=4.16 Hz, 1 F) −109.78 (br s, 1 F).

### Q14: 4-[[9-[(3S)-Tetrahydrofuran-3-yl]-8-(2,4,6-trifluoroanilino)purin-2-yl]amino]cyclohexyl] N,N-dimethylcarbamate

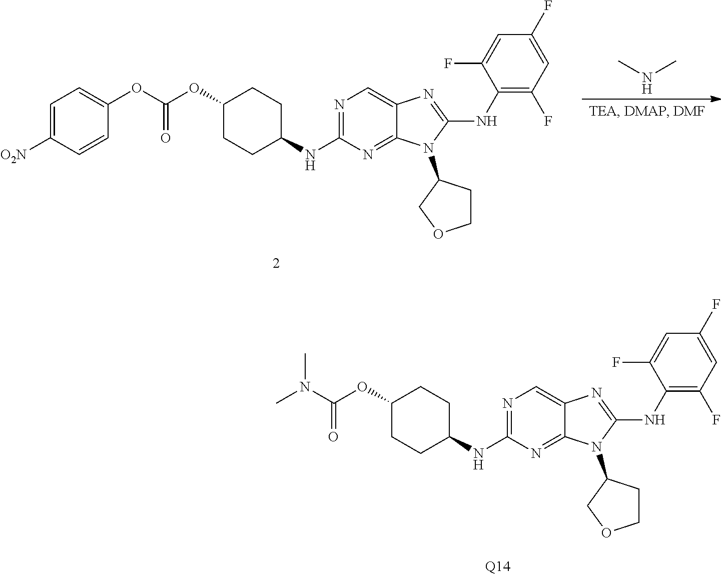

A mixture of (4-nitrophenyl) [4-[[9-[(3S)-tetrahydrofuran-3-yl]-8-(2,4,6-trifluoroanilino)purin-2-yl]amino]cyclohexyl]carbonate (250 mg, 407.47 umol, 1 eq), N-methylmethanamine;hydrochloride (66.45 mg, 814.94 umol, 2 eq), TEA (206.16 mg, 2.04 mmol, 283.57 uL, 5 eq), DMAP (4.98 mg, 40.75 umol, 0.1 eq) in DCM (4 mL) was degassed and purged with N₂ for 3 times, and then the mixture was stirred at 20° C. for 12 h under N₂ atmosphere. LCMS showed the reaction was complete and the desired mass was detected. The mixture was concentrated to get a crude product. The residue was purified by prep-HPLC (neutral condition, column: Waters Xbridge BEH C18 100*25 mm*5 um;mobile phase: [water(10 mM NH₄HCO₃)-ACN];B %: 30%-55%,10 min) to give [4-[[9-[(3S)-tetrahydrofuran-3-yl]-8-(2,4,6-trifluoroanilino)purin-2-yl]amino]cyclohexyl] N,N-dimethylcarbamate (Q14) (66 mg, 126.52 umol, 31.05% yield, 99.593% purity) as a white solid.

LC-MS: 520.3 [M+1]⁺; ¹⁹F NMR (377 MHz, CHLOROFORM-d) δ ppm-115.72 (br d, J=5.55 Hz, 1 F) −109.86 (br d, J=4.16 Hz, 1 F); ¹H NMR (400 MHz, CHLOROFORM-d) δ ppm 1.23-1.47 (m, 2H) 1.50-1.71 (m, 2H) 1.99-2.19 (m, 4H) 2.26-2.41 (m, 1H) 2.61-2.80 (m, 1H) 2.91 (br s, 6H) 3.73-3.83 (m, 1H) 3.86-3.98 (m, 2H) 4.28-4.57 (m, 2H) 4.58-4.88 (m, 2H) 5.43-5.64 (m, 1H) 6.79 (br t, J=8.00 Hz, 2H) 7.90 (s, 1H) 8.27 (s, 1H).

### Q15: Trimethyl-[2-oxo-2-[4-[[9-[(3S)-tetrahydrofuran-3-yl]-8-(2,4,6-trifluoroanilino)purin-2-yl]amino]cyclohexoxy]ethyl]ammonium

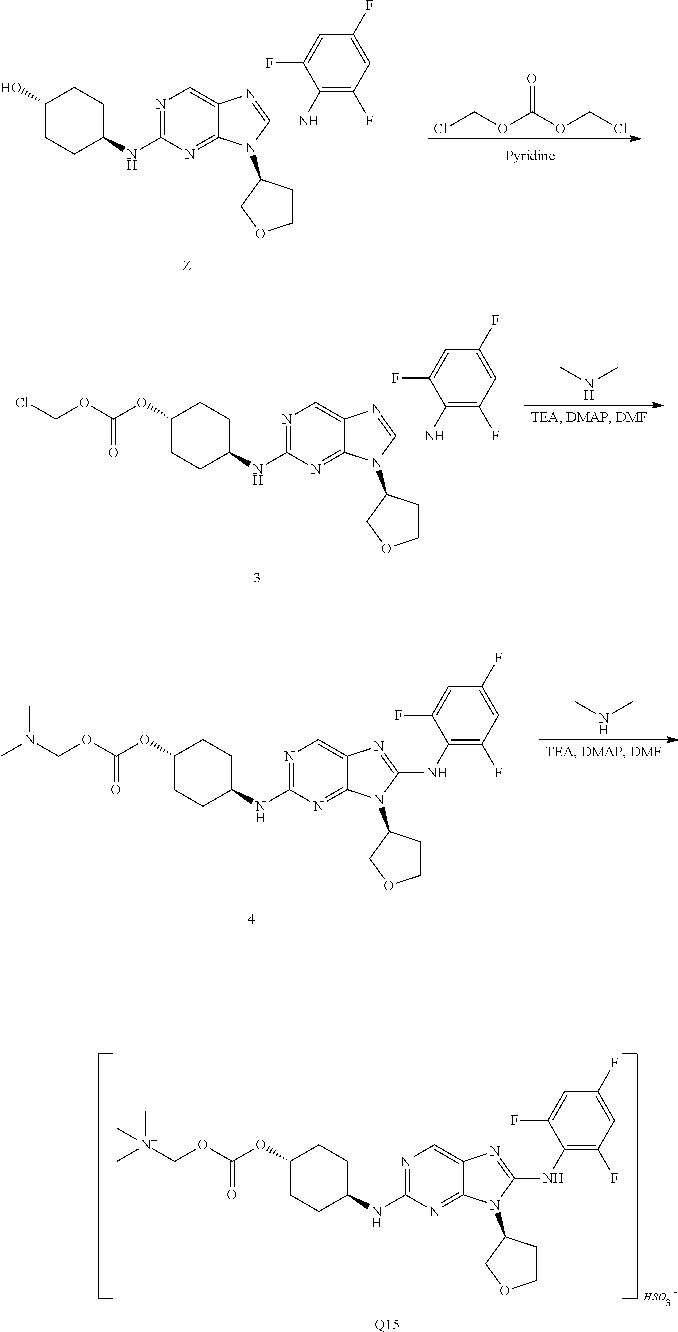

### Step 1: [4-[[9-[(3S)-tetrahydrofuran-3-yl]-8-(2,4,6-trifluoroanilino)purin-2-yl]amino]cyclohexyl]2-chloroacetate

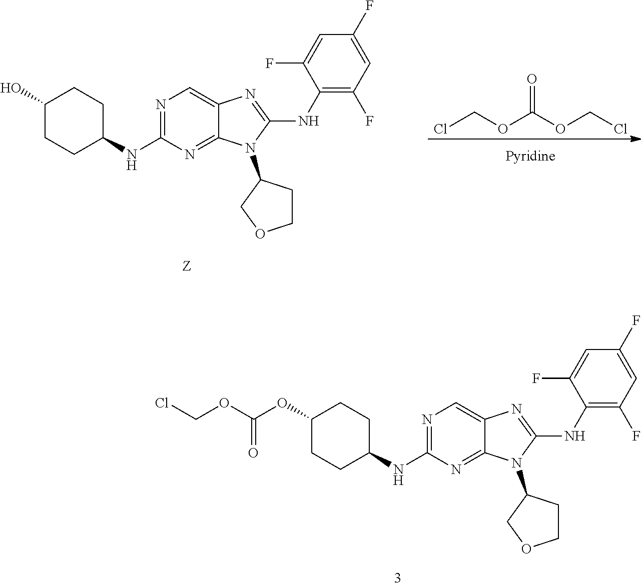

A mixture of 4-[[9-[(3S)-tetrahydrofuran-3-yl]-8-(2,4,6-trifluoroanilino)purin-2-yl]amino]cyclohexanol (0.5 g, 1.11 mmol, 1 eq) and (2-chloroacetyl) 2-chloroacetate (285.96 mg, 1.67 mmol, 1.5 eq) in Py (5 mL) was stirred at 20° C. for 1 h. LCMS showed Z was almost consumed and desired mass was detected. The reaction mixture was concentrated to give [4-[[9-[(3S)-tetrahydrofuran-3-yl]-8-(2,4,6-trifluoroanilino)purin-2-yl]amino]cyclohexyl]2-chloroacetate (585 mg, crude) as brown oil.

LC-MS: 525.1 [M+1]⁺

### Step 2: [4-[[9-[(3S)-tetrahydrofuran-3-yl]-8-(2,4,6-trifluoroanilino)purin-2-yl]amino]cyclohexyl]2-(dimethylamino)acetate

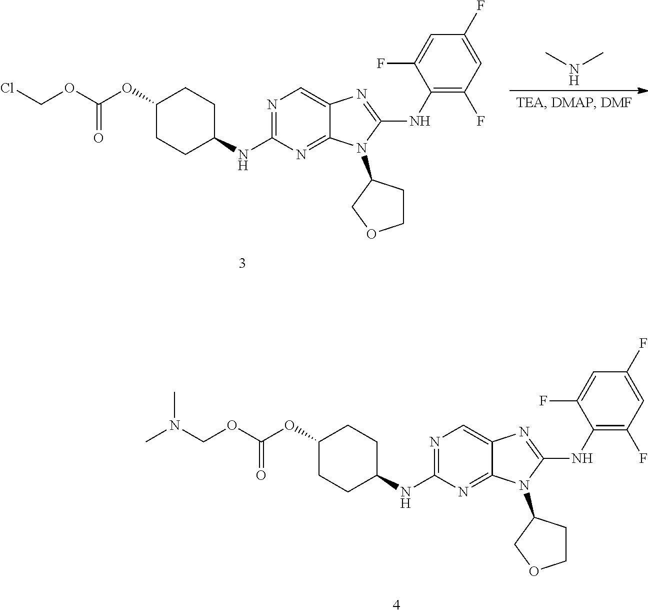

To a mixture of [4-[[9-[(3S)-tetrahydrofuran-3-yl]-8-(2,4,6-trifluoroanilino) purin-2-yl]amino]cyclohexyl]2-chloroacetate (585 mg, 1.11 mmol, 1 eq) and N-methylmethanamine;hydrochloride (181.75 mg, 2.23 mmol, 2 eq) in DCM (7 mL) was added TEA (451.08 mg, 4.46 mmol, 620.47 uL, 4 eq) and DMAP (13.62 mg, 111.45 umol, 0.1 eq), then the mixture was stirred at 20° C. for 12 h. LCMS showed 3 was almost consumed and desired mass was detected. The reaction mixture was concentrated to give the crude product. The crude product was purified by prep-TLC (SiO₂, Ethyl acetate/MeOH=5/1, Rt=0.5) to give [4-[[9-[(3S)-tetrahydrofuran-3-yl]-8-(2,4,6-trifluoroanilino)purin-2-yl]amino]cyclohexyl]2-(dimethylamino)acetate (500 mg, crude) as yellow oil.

LC-MS: 534.4 [M+1]⁺.

### Step 3: trimethyl-[2-oxo-2-[4-[[9-[(3S)-tetrahydrofuran-3-yl]-8-(2,4,6-trifluoroanilino)purin-2-yl]amino]cyclohexoxy]ethyl]ammonium

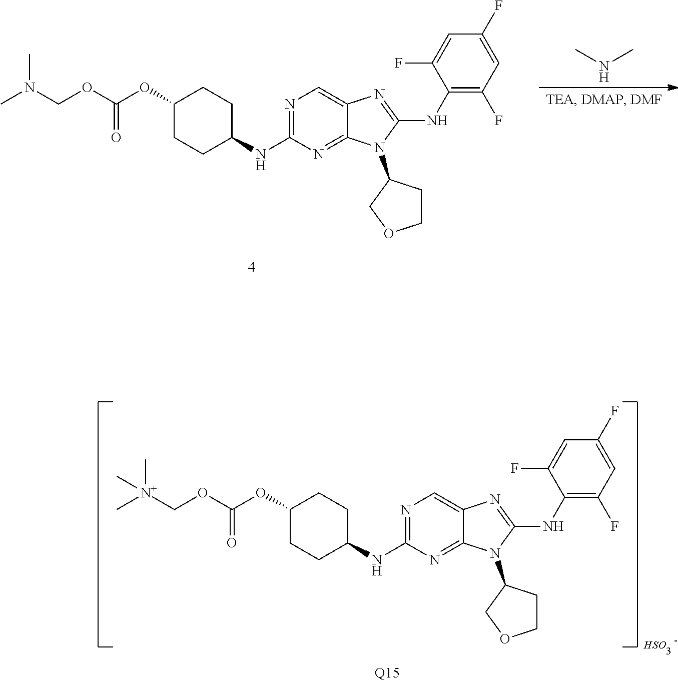

To a solution of [4-[[9-[(3S)-tetrahydrofuran-3-yl]-8-(2,4,6-trifluoroanilino)purin-2-yl]amino]cyclohexyl]2-(dimethylamino)acetate (260 mg, 487.31 umol, 1 eq) in MeCN (3 mL) was added dimethyl sulfate (61.46 mg, 487.31 umol, 46.21 uL, 1 eq) at 20° C., then the mixture was stirred at 20° C. for 0.5 h. LCMS showed the reaction was complete and desired mass was detected. The reaction mixture was quenched by TEA (0.05 mL) and then concentrated to give the crude product. The crude product was purified by prep-HPLC (FA condition, column: Phenomenex Luna C18 150*30 mm*5 um;mobile phase: [water(FA)-ACN]; B %: 1%-25%, 8 min) to give trimethyl-[2-oxo-2-[4-[[9-[(3S)-tetrahydrofuran-3-yl]-8-(2,4,6-trifluoroanilino)purin-2-yl]amino]cyclohexoxy]ethyl]ammonium (Q15) (62 mg, 103.12 umol, 21.16% yield, 98.896% purity, FA) as a yellow solid.

LC-MS: 548.2 [M]⁺; ¹H NMR (400 MHz, CHLOROFORM-d) δ ppm 1.20-1.48 (m, 2H) 1.63 (br d, J=11.01 Hz, 2H) 1.96-2.12 (m, 2H) 2.21 (br d, J=11.01 Hz, 2H) 2.27-2.35 (m, 1H) 2.61-2.80 (m, 1H) 3.30-3.65 (m, 9H) 3.71-3.89 (m, 3H) 3.91-4.07 (m, 1H) 4.34-4.55 (m, 2H) 4.77-4.99 (m, 3H) 5.56 (br s, 1H) 6.78 (br t, J=8.00 Hz, 2H) 8.22 (s, 1H) 8.71 (br s, 1H); ¹⁹F NMR (377 MHz, CHLOROFORM-d) δ ppm-115.78 (br s, 1 F) −110.46-−109.48 (m, 1 F) Q16: [(8Z)-2-[(4-Hydroxycyclohexyl)amino]-9-f(3S)-tetrahydrofuran-3-yl]-8-(2,4,6-trifluorophenyl)imino-purin-7-ylmethyl acetate

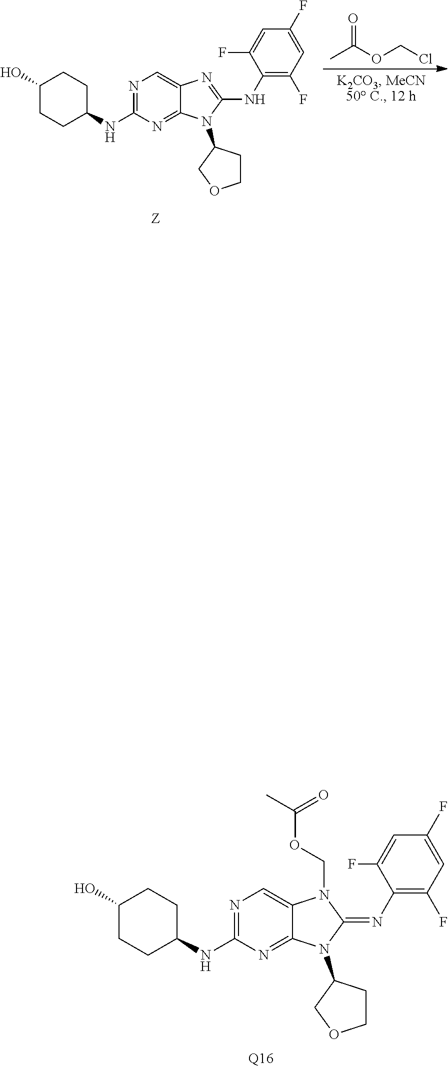

### Step 1: [(8Z)-2-[(4-hydroxycyclohexyl)amino]-9-[(3S)-tetrahydrofuran-3-yl]-8-(2,4,6-trifluorophenyl)imino-purin-7-yl]methyl acetate

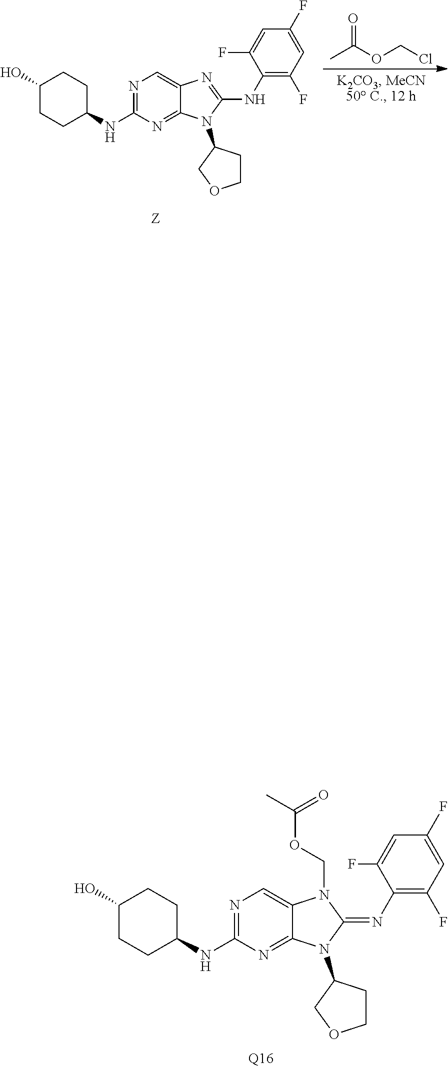

To a solution of 4-119-|(3S)-tetrahydrofuran-3-yl]-8-(2,4,6-trifluoroanilino)purin-2-yl]amino]cyclohexanol (300 mg, 668.98 umol, 1 eq) in MeCN (4 mL) was added K₂CO₃ (184.91 mg, 1.34 mmol, 2 eq) and chloromethyl acetate (72.60 mg, 668.98 umol, 98.40 uL, 1 eq). The mixture was stirred at 50° C. for 12 h. LCMS showed the reaction was complete and desired mass was detected. The reaction mixture was filtered and then the filtrate was concentrated to give the crude product. The crude product was purified by prep-HPLC (basic condition, column: Waters Xbridge Prep OBD C_{18 150*40} mm*10 um; mobile phase: [water(NH₃H₂O+NH₄HCO₃)-ACN]; B %: 20%-50%, 8 min), then it was product purified again by prep-HPLC (FA condition, column: Phenomenex Luna C18 75*30 mm*3 um;mobile phase: [water(FA)-ACN];B %: 30%-65%,8 min) to give [(8Z)-2-[(4-hydroxycyclohexyl)amino]-9-[(3S)-tetrahydrofuran-3-yl]-8-(2,4,6-trifluorophenyl)imino-purin-7-yl]methyl acetate (Q16) (63 mg, 120.82 umol, 18.06% yield, 99.817% purity) as a white solid.

LC-MS: 521.1 [M+1]⁺; ¹H NMR (400 MHz, CHLOROFORM-d) δ ppm 1.20-1.35 (m, 2H) 1.38-1.53 (m, 2H) 2.00 (s, 3H) 2.04 (br d, J=3.13 Hz, 1H) 2.11 (br s, 4H) 2.27 (ddt, J=12.59, 10.05, 7.63, 7.63 Hz, 1H) 2.65-2.79 (m, 1H) 3.60-3.80 (m, 2H) 3.99 (td, J=7.97, 5.19 Hz, 1H) 4.06 (t, J=8.38 Hz, 1H) 4.14-4.23 (m, 1H) 4.26 (q, J=7.75 Hz, 1H) 4.99-5.10 (m, 1H) 5.16-5.28 (m, 1H) 5.41 (s, 2H) 6.69 (br t, J=8.19 Hz, 2H) 7.87 (s, 1H). ¹⁹F NMR (376 MHz, CHLOROFORM-d) δ ppm-120.26 (br s, 1 F) −117.16 (s, 1 F).

### Q17: [(8Z)-2-[(4-hydroxycyclohexyl)aminol-9-[(3S)-tetrahydrofuran-3-yl]-8-(2,4,6-trifluorophenyl)imino-purin-7-yl]methyl phosphono hydrogen phosphate

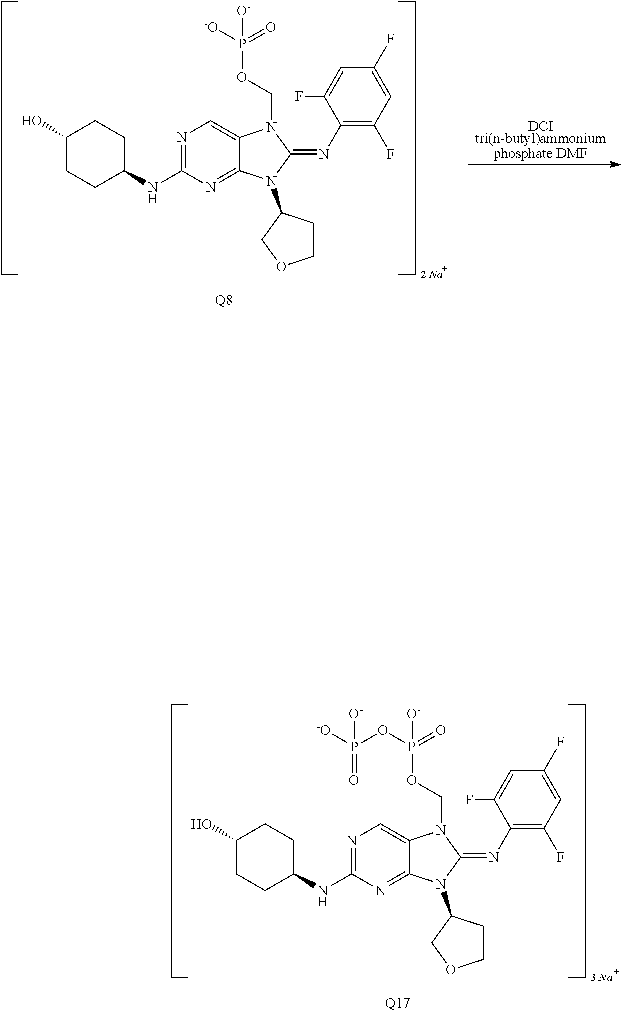

To a solution of [[(8Z)-2-[(4-hydroxycyclohexyl)amino]-9-[(3S)-tetrahydrofuran-3-yl]-8-(2,4,6-trifluorophenyl)imino-purin-7-yl]methoxy-sodiooxy-phosphoryl]oxysodium (0.2 g, 327.58 umol, 98.668% purity, 1 eq) in DMF (2 mL)was added CDI (212.47 mg, 1.31 mmol, 4 eq), the mixture was stirred at 20° C. for 2 h. LCMS showed the reaction was complete and desired intermediate was detected. Excess of CDI was eliminated by addition of MeOH (83.96 mg, 2.62 mmol, 106.04 uL, 8 eq) and the mixture was stirred for 30 min. Then tributyl (phosphonooxy) ammonium (1 M, 3.28 mL, 10 eq) were added. The mixture was stirred for another 12 h. LCMS showed the reaction was complete and desired mass was detected. The reaction mixture was concentrated to give the crude product. The crude product was purified by prep-HPLC (basic condition, column: Waters Xbridge Prep OBD C18 150*40 mm*10 um; mobile phase: [water (NH₃H₂O+NH₄HCO₃)-ACN];B %: 1%-30%,8 min). Then it was exchange to sodium salt by sodium ion exchange resin, the crude product was purified again by prep-HPLC (column: Waters Xbridge BEH C18 100*30 mm*10 um; mobile phase: [Water-ACN]; B %: 1%-20%, 8 min) to give [(8Z)-2-[(4-hydroxycyclohexyl)amino]-9-[(3S)-tetrahydrofuran-3-yl]-8-(2,4,6-trifluorophenyl)imino-purin-7-yl]methyl phosphono hydrogen phosphate (Q17) (25 mg, 39.16 umol, 11.95% yield, 100% purity) as a white solid.

LC-MS: 639.2 [M+1]⁺; ¹H NMR (400 MHz, DEUTERIUM OXIDE) δ ppm 1.17-1.52 (m, 4H) 1.89-2.15 (m, 4H) 2.20-2.41 (m, 1H) 2.49-2.69 (m, 1H) 3.50-3.80 (m, 2H) 3.85-4.08 (m, 2H) 4.16-4.44 (m, 2H) 5.06 (br s, 1H) 5.34 (br d, J=6.13 Hz, 2H) 6.90 (br t, J=8.63 Hz, 2H) 8.08 (br s, 1H); ¹⁹F NMR (377 MHz, DEUTERIUM OXIDE) δ ppm-121.21-−120.59 (m, 1 F) −116.57 (br s, 1 F); ¹P NMR (162 MHz, DEUTERIUM OXIDE) δ ppm-13.52 (br d, J=21.80 Hz, 1 P) −6.17 (br d, J=21.80 Hz, 1 P).

### Example 2

The solubilities of compounds of the invention in PBS were measured and compared with known compound Z.

### Thermodynamic Solubility Assay

Solubility Study: The thermodynamic solubility assay employed was the shake flask method followed by HPLC analysis. The method prepared a saturated solution of the compound, followed by an assay of the solution using HPLC and UV detection. Saturated solutions were prepared in PBS buffer (0.01 M phosphate buffer, 0.0027 M potassium chloride, and 0.137 M sodium chloride, pH 7.4). About 18 mg compound powder was weighed and placed in a 2 mL glass vial, then PBS buffer was introduced into the vial to obtain a 30-60 mg/mL target concentration. This solution was vortexed for 2 minutes. The solutions were incubated and shook on an orbital shaker for 24 hr at room temperature (25±2° C.). About 500 μL solubility solution was transferred into a new MultiScreen filter plate (Membrane of polycarbonate), filtered by millipore vacuum manifold, and collected in the filtrate as the TS solution. The filtrate was diluted with PBS buffer by a factor of 20 fold to make it diluent. Three UV standard solutions (20, 200, 2000 μg/mL) were injected into HPLC from low to high concentration, followed by testing of the diluent. Testing samples were injected in duplicate. The filtrate was analyzed by HPLC equipped with UV, and the solubility was calculated based on acquired peak areas and dilution factors with a standard external method.

### Thermodynamic Solubility Results:

The thermodynamic solubility of the compounds of the invention in PBS as compared to known compound Z are presented in Table 1.

TABLE 1

| Compound | Thermodynamic Solubility |
|---|---|
| Number | in PBS, mg/mL |
| Q7 | 29.99 |
| Q8 | 45.56 |
| Z | 0.06063 |
| Q9 | 19.44 |
| Q10 | 0.07521 |
| Q11 | <0.020 |
| Q12 | 29.55 |
| Q13 | <0.020 |
| Q14 | <0.020 |
| Q15 | 17.34 |
| Q16 | 0.1138 |

Animals (such as humans) undergoing a surgical procedure may need compounds administered intravenously. Known compound Z has a solubility in PBS of 0.06063 mg/mL. This low solubility effectively prevents its intravenous administration.

11 compounds were synthesized and tested for their solubility in PBS. 7 of the 11 compounds had improved solubility in PBS over known compound Z. The degree of improvement in solubility for these 7 compounds over known compound Z is presented in Table 2:

TABLE 2

| Compound | Degree of improvement in |
|---|---|
| Number | solubility over Z |
| Q7 | 494x |
| Q8 | 751x |
| Q9 | 320x |
| Q10 | 1.24x |
| Q12 | 487x |
| Q15 | 285x |
| Q16 | 1.87x |

The addition of an ammonium or phosphate substituent improved the solubility by at least 285-fold over known compound Z. In particular, the addition of a phosphate substituent improved solubility by at least 320-fold over known compound Z. Q8 demonstrates a 751-fold improvement in solubility over known compound Z. This large and unexpected improvement in solubility allows these compounds to be administered intravenously, for example, before, during or after a surgical procedure.

### Example 3

Rapid cleavage of the compounds of the invention into compound Z in the subject was sought to reduce exposure to the subject of the compounds of the invention and to increase exposure to the subject of compound Z. Rat pharmacokinetic studies were conducted to measure cleavage of the compounds of the invention in vivo.

### In Vivo Rat Pharmacokinetic Studies

Rat pharmacokinetic study samples were used for PK characterization. Male Sprague-Dawley rats weighing 150-300 g were intraperitoneal (ip), intravenous (i.v.) dosed formulated in PBS, pH 7.4. The blood samples at several times post dosing were collected and advanced to plasma preparation with K₂EDTA as an anticoagulant.

The blank plasma samples used for the PK characterization control were prepared from undosed rat groups. After IV administration, the amount of parent compound remaining at 0.5 hours after dosing was used as a measure of the cleavage rate. After IP administration, the amount of parent compound remaining at 2 hours after dosing was used as a measure of the cleavage rate. We sought compounds with >90% cleavage.

### Results:

The in vivo study results are provided in Table 3:

TABLE 3

| | Compound | Compound | Compound | Compound |
|---|---|---|---|---|
| | Qx | Z | Qx | Z |
| | Present in | Present in | Present in | Present in |
| | Plasma at | Plasma at | Plasma at | Plasma at |
| | 0.5 hr | 0.5 hr | 2 hr | 2 hr |
| | After i.v. | After i.v. | After i.p. | After i.p. |
| | dose\hat{ } | dose\hat{ } | dose^{$} | dose^{$} |
| Compound | (ng/mL)* | (ng/mL)* | (ng/mL)* | (ng/mL)* |
| Q7 | 13 | 232 | 36 | 83.5 |
| Q8 | 21 | 244 | 12^{#} | 183^{#} |
| Q9 | BQL | 252 | BQL | 56.1 |
| Q12 | nt | nt | 214 | 9.2 |
| Q15 | nt | nt | 125 | 26.5 |

All results linearly scaled to 2 mg/kg equivalent dose. Not Tested, n.t.; Below quantification limit, BQL.
\hat{ }Compounds Q7, Q8 and Q9 administered via i.v. and studied at 1 mg/kg formulated in PBS.
^{$}Compounds Q7, Q9, Q12, and Q15 administered via i.p. and studied at 2 mg/kg, formulated in PBS.
^{#}Compound Q8 administered via i.p. and studied at 29.8 mg/kg formulated in PBS.

Our original expectation was that all 5 of the tested compounds would be rapidly cleaved in vivo. The PK studies revealed a different outcome. Compound Q12 and compound Q15 were cleaved very slowly, with 80-90% of the compound present at 2 hrs post-dosing. Compound Q7 was moderately slowly cleaved, with 30% still present at 2 hr. Both compound Q8 and compound Q9 exhibited rapid cleavage, above 90% at 2 hr.

Identifying compounds with rapid cleavage in vivo was unpredictable and required the synthesis and testing of multiple types of compounds to identify compounds with the desired properties.

### Example 4

Testing the Effect of Compound 08 on Kidney Function after Reperfusion Injury

Reperfusion injury Many life events and conditions, such as trauma and surgery, can create ischemia and reperfusion injury in organs such as the heart, brain, liver, and kidney. Among the reperfusion injuries, acute kidney injury (AKI) during cardiac surgery is a significant cause of death and long-term dialysis. During these surgeries lasting from 2-6 hours, subjects can be placed on cardiac bypass machines, which can result in low blood flow to the kidney. Once blood flow is restored, reperfusion of the kidney can lead to reperfusion cellular apoptosis strongly focused on the highly metabolically active-mitochondria-rich proximal renal tubule epithelial cells (RTE). Damage to the proximal tubule epithelial cells can lead to kidney dysfunction. Kidney dysfunction can result as the tubules stop the excretion of products such as creatinine and reabsorption of metabolites and ions.

Renal tubular cell injury can lead to apoptosis and necrosis and rapid death of RTE cells. This can lead to kidney dysfunction. Dead cells can also lead to inflammation and tubular damage. Inflammation, tubular damage, and regeneration imbalance can lead to fibrosis. If regeneration does not occur and the tubular damage is not mitigated chronic kidney dysfunction can occur and the kidney can develop fibrosis.

Kidney function experimental. Kidney reperfusion injury is often called acute kidney injury, AKL The rat bilateral short-warm kidney ischemia-reperfusion system (Grynberg et al. Am J Pathol 191:817-828, 2021) was used to study the effect of compounds of the invention on kidney reperfusion injury, or AKL This study of compound Q8 demonstrated that compound Q8 significantly reduces kidney dysfunction, tubular injury, and fibrosis.

Brief description of AKI Study: The study examined rats given oral compound Q8 three times at −1 hour, +10 hours, and 24 h post-lesion, or nine times over four days, at −1 hr, +10 hours and every morning and evening. The dose chosen was 103 mg/kg (0.134 mmol/kg). Control groups include a sham operation group receiving drug vehicle, peritoneum open but no ligature application to the kidney artery. A vehicle group in which the artery ligature was applied served as the lesion group. The dose regimen was chosen based on pilot pharmacokinetic studies; these studies showed that the dose method would achieve compound Z exposure above 3.4 uM (active drug species) for more than 16 consecutive hours of each day. A 3.4 uM plasma concentration of compound Z (1560 ng/mL, 3.4 uM) was chosen because this amount achieved efficacy in studies measuring the suppression of cJun phosphorylation in human skin (van der Velden et al. Clinical and translational medicine, 5(1), pp. 1-18). This chosen amount is also between the IC₉₅ and the IC₅₀ inhibition of Jnk activity in cell extracts and human PBMCs (Krenitsky et al. Bioorganic & Medicinal Chemistry Letters, 22(3), pp. 1433-1438). It is also similar to the oral C_{avg} exposure of compound Z at 60 mg/kg, which is an effective dose of compound Z in rat models of AKI (Grynberg et al. Am J Pathol 191:817-828, 2021).

### Methods for Measurement of Kidney Function, Tubular Damage, and Fibrosis.

Rat bilateral short-warm kidney ischemia-reperfusion injury (“IRI”) system: Male outbred Sprague-Dawley rats (approximately 300 g from the Monash Animal Research Platform), six/group, were anesthetized using ketamine/xylazine, and surgery performed with both renal pedicles clamped for 25 minutes (ischemia) then released (reperfusion) as previously described (Grynberg et al. Am J Pathol 191:817-828, 2021). Blood samples were collected from the tail vein during the experiment, and blood was collected by cardiac puncture at the killing time. The Monash Medical Centre Animal Ethics Committee approved animal studies.

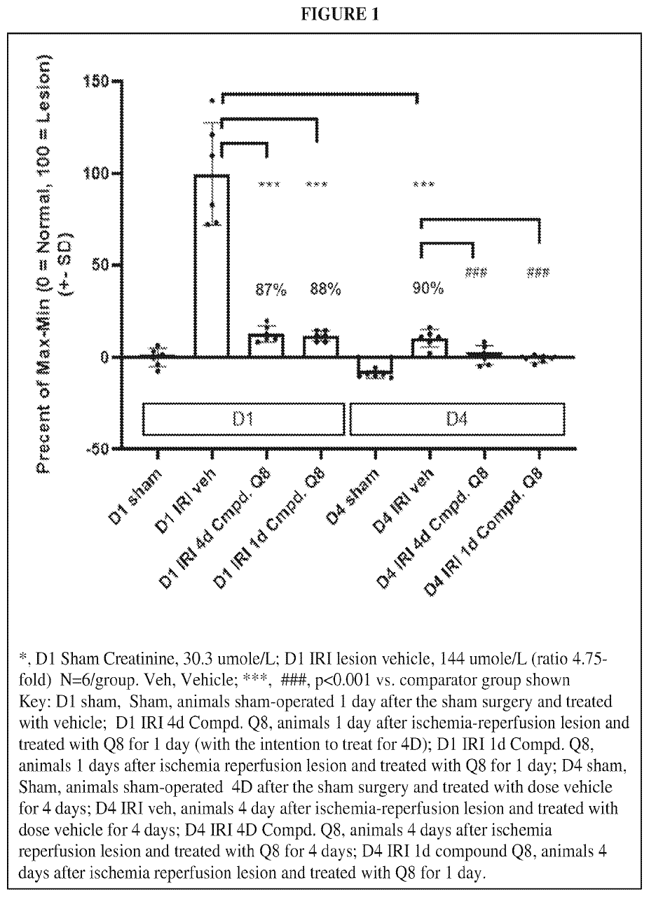

Biochemistry: FIG. 1 shows plasma creatinine levels measured with an ARL Analyser (Dupont, Wilmington, DE).

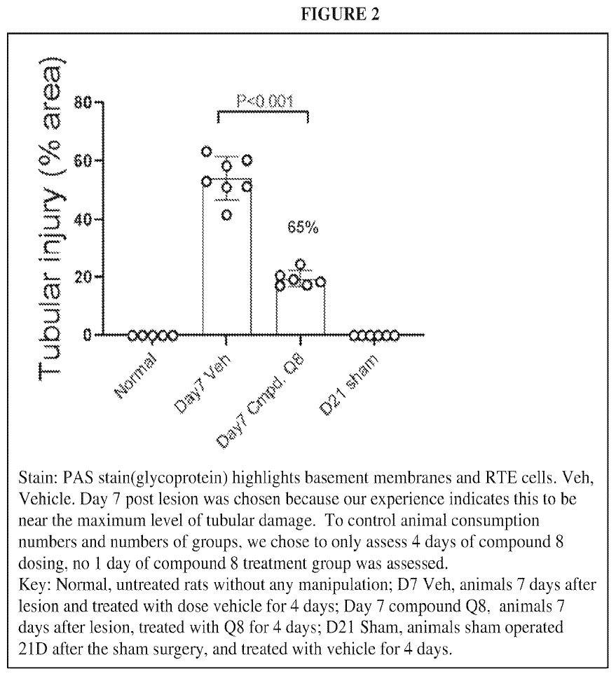

Tubular damage and inflammation scores: FIG. 2 shows the percentage of tubular cross-sections exhibiting damage in the outer medulla was assessed on periodic acid Schiff (PAS) stained kidney sections as previously described (Grynberg et al. Am J Pathol 191:817-828, 2021). Analysis was performed on blinded slides.

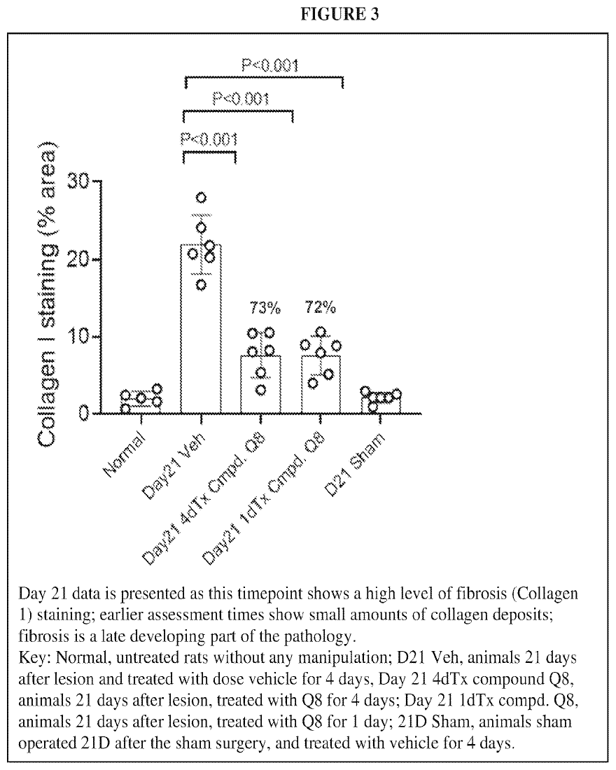

Immunohistochemistry: FIG. 3 shows the degree of fibrosis in the kidney through immunoperoxidase staining of Carnoy-fixed, paraffin-embedded kidney tissue sections using a 3-layer avidin-biotin complex (ABC) based method (Hou et al. Clin Exp Pharmacol Physiol 45:250-260, 2017). Staining used rabbit monoclonal antibodies recognizing collagen I (E8F4L, 1 in 400 dilutions; Cell Signaling Technology, Danvers, MA, USA), followed by biotinylated rabbit anti-rat IgG (BA-400, 1 in 500 dilutions; Thermo Fisher, Waltham, MA, USA), and the VECTASTAIN® Elite® ABC-HRP Kit (PK-6100). The interstitial collagen I deposition area was assessed in the outer medulla and cortex by image analysis, avoiding medium and large arteries, using cellSens software V1.18 (Olympus, Notting Hill, Victoria, Australia) as previously described (Leong et al. Toxins 13:700, 2021). Analysis was performed on blinded slides.

Statistical Analysis: Data are shown as means±1 SD. Data were analyzed by one-way analysis of variance with the Tukey multiple comparison tests using GraphPad Prism 9.0 software (GraphPad Software, San Diego, CA).

Kidney dysfunction is the most immediately measurable outcome of kidney reperfusion injury. It can often be seen with 8-24 hours after the insult in animals and humans. FIG. 1 presents data showing the effect of treatment with compound Q8 on kidney dysfunction, assessed by plasma creatinine levels, measured at 1 day and 4 days post-lesion.

Discussion and Conclusion. FIG. 1 shows that compound Q8 can be delivered at doses which reduce serum/plasma creatinine levels by 87-88%. This demonstrates that the kidneys are removing creatinine from the blood, which demonstrates reduction in kidney dysfunction. Compound Q8 suppresses kidney dysfunction by 87-88%. This level of kidney dysfunction reduction was achieved with three doses of compound Q8.

Thus, compound Q8 reduces kidney dysfunction in this rat short-warm ischemia kidney injury model. Compound Q8 therefore can treat the dysfunction symptom of ischemic reperfusion.

Tubular Damage: Inflammation and tubular damage are significant negative outcomes of IRI in the kidney and other organs. FIG. 2 presents data on the effect to kidney tubular damage by treatment with compound Q8 as measured at 7 days post-lesion. The percentage of tubular cross-sections exhibiting damage in the outer medulla was assessed on periodic acid Schiff (PAS) stained kidney section. PAS staining highlights basement membranes and RTE cells, this method has been found to be the best way to visualize the loss of tubular cellularity and disruption of normal structure.

Discussion and Conclusion. Compound Q8 reduces tubular damage by 65% on D7 in the rat bilateral warm short ischemia model. Compound Q8 therefore can treat ischemic reperfusion.

Fibrosis: Fibrosis is a significant negative outcome of ischemia-reperfusion injury in the kidney and other organs. It leads to long-term kidney dysfunction and can lead to the need for dialysis, transplant, or death. FIG. 3 presents data showing the effect of treatment with compound Q8 on kidney fibrosis measured at 21 days post-lesion.

Discussion and Conclusion. Q8 reduces fibrosis component Coll histological staining by 73% on D21 in the rat two kidney warm short ischemia model. This finding shows that compound Q8 treats the fibrosis that results from the lesion.

The data in Example 4 demonstrates that compound Q8 treats kidney dysfunction, tubular damage and fibrosis. Compound Q8 can therefore treat conditions associated with reperfusion injury, such as injuries to the kidney. Compound Q8 may also find application to lung, brain, and liver reperfusion injury caused by trauma and surgery.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure.

At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof are encompassed by the disclosure unless otherwise indicated herein or otherwise clearly contradicted by context.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1. A compound, or a salt or a tautomer or an isomer thereof, having a structure according to formula (I) or (II): 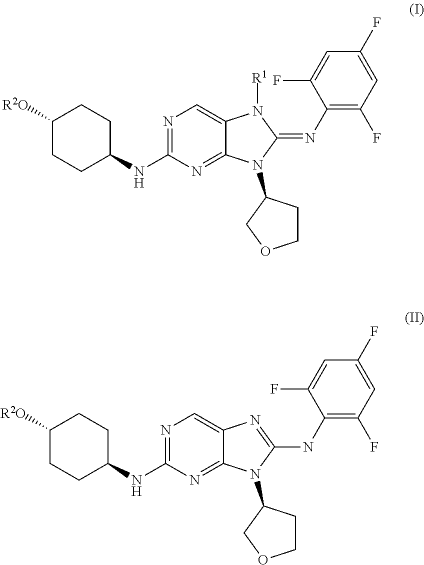
wherein
R¹, when present, is C(O)R^{a} or substituted or unsubstituted C₁₋₆ alkylene
wherein R^{a} is substituted or unsubstituted C₁₋₆ alkoxy or substituted or unsubstituted C₁₋₆ alkylamino
R² is H or C(O)R^{b} or substituted or unsubstituted phosphate
wherein R^{b} is substituted or unsubstituted C₁₋₆ alkyl or substituted or unsubstituted C₁₋₆ alkoxy or substituted or unsubstituted C₁₋₆ alkylamino
with the proviso that the compound is not

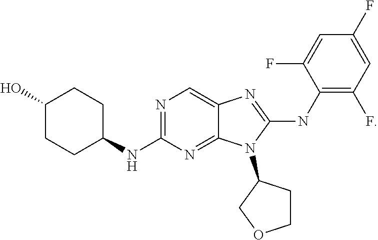 [SMILES: O[C@H]1CC[C@H](Nc2ncc3nc(Nc4c(F)cc(F)cc4F)n([C@H]4CCOC4)c3n2)CC1]

2. The compound of claim 1, or a salt or a tautomer or an isomer thereof, wherein R² is H, C(O)CH₃, C(O)CH₂CH₃, C(O)CH(CH₃)₂, C(O)CH₂OH, C(O)CH₂NH₂, C(O)N(CH₃)₂, C(O)CH(NH₂)CH₃, C(O)OCH₂CH₃, C(O)O(CH₂)₂N(CH₃)₃, C(O)CH₂N(CH₃)₃, or substituted or unsubstituted phosphate.

3. The compound of claim 1, or a salt or a tautomer or an isomer thereof, wherein R² is H.

4. The compound of claim 2, or a salt or a tautomer or an isomer thereof, wherein R² is 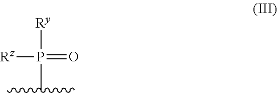
wherein RV and R^{z} are each individually selected from OH, O-phosphate, or C₁₋₆ alkoxy.

5. The compound of claim 2, or a salt or a tautomer or an isomer thereof, wherein R² is 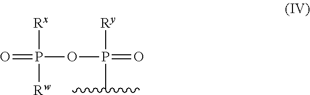
wherein R^{w}, R^{x}, and R^{y} are each individually selected from OH, O-phosphate, or C₁₋₆ alkoxy.

6. The compound of claim 2, or a salt or a tautomer or an isomer thereof, wherein R² is 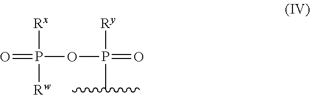
wherein R^{w}, R^{x}, and R^{y} are each individually selected from OH or C₁₋₆ alkoxy.

7. The compound of claim 2, or a salt or a tautomer or an isomer thereof, wherein R² is

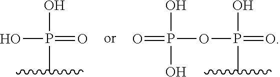 [SMILES: *P(=O)(O)O ; *P(=O)(O)OP(=O)(O)O]

8. The compound of claim 1, or a salt or a tautomer or an isomer thereof, wherein the structure is according to formula (II).

9. The compound of claim 1, or a salt or a tautomer or an isomer thereof, wherein the structure is according to formula (I), and R¹ has a structure according to formula (V): 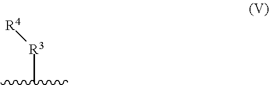
wherein R³ is C═O, CH₂, CH(CH₃), CH(CH₂CH₃), CH(CH(CH₃)₂), or CH(CH₂OH),
when R³ is C═O, then R⁴ is unsubstituted C₁₋₆ alkyl, —OR⁵, or NR⁵R⁶,
wherein R⁵ and R⁶ are each individually selected from unsubstituted C₁₋₆ alkyl or C₁₋₆ alkyl substituted with a trialkylammonium moiety
when R³ is CH₂, CH(CH₃), CH(CH₂CH₃), CH(CH(CH₃)₂), or CH(CH₂OH), then R⁴ is substituted or unsubstituted phosphate.

10. The compound of claim 1, or a salt or a tautomer or an isomer thereof, wherein the structure is according to formula (I), and R¹ has a structure according to formula (V): 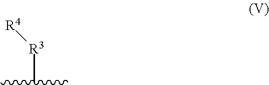
wherein R³ is CH₂, CH(CH₃), CH(CH₂CH₃), CH(CH(CH₃)₂), or CH(CH₂OH), and R⁴ is substituted or unsubstituted phosphate.

11. The compound of claim 10, or a salt or a tautomer or an isomer thereof, wherein R⁴ is 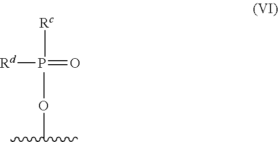
wherein R^{c} and R^{d} are each individually selected from OH, O-phosphate, or C₁₋₆ alkoxy.

12. The compound of claim 10, or a salt or a tautomer or an isomer thereof, wherein R⁴ is 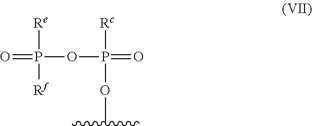
wherein R^{c}, R^{e}, and R^{f} are each individually selected from OH, O-phosphate, or C₁₋₆ alkoxy.

13. The compound of claim 10, or a salt or a tautomer or an isomer thereof, wherein R⁴ is 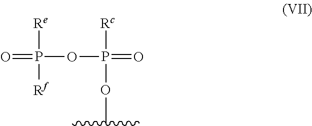
wherein R^{c}, R^{e}, and R^{f} are each individually selected from OH or C₁₋₆ alkoxy.

14. The compound of claim 10, or a salt or a tautomer or an isomer thereof, wherein R⁴ is

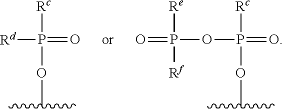

15. The compound of claim 3, or a salt or a tautomer or an isomer thereof, wherein R³ is CH₂.

16. The compound of claim 1, or a salt or a tautomer or an isomer thereof, having a structure which is

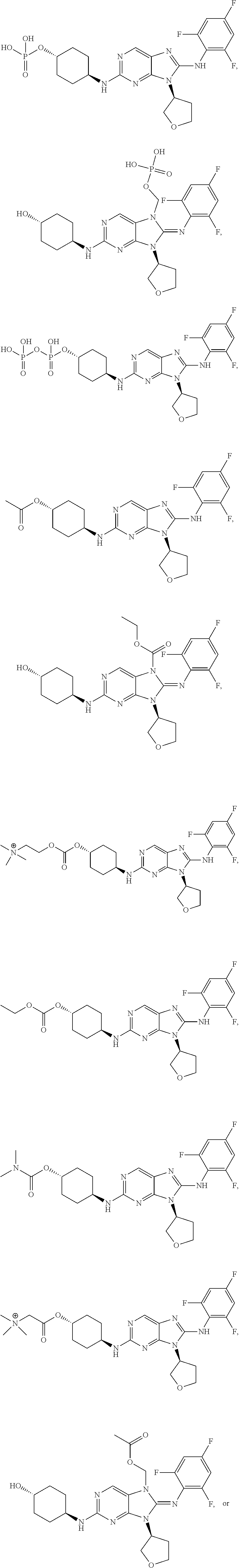

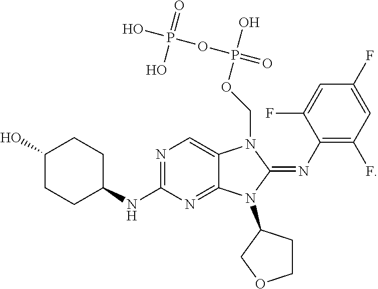 [SMILES: O=P(O)(O)OP(=O)(O)OCn1/c(=N\c2c(F)cc(F)cc2F)n([C@H]2CCOC2)c2nc(N[C@H]3CC[C@H](O)CC3)ncc21]

17. The compound of claim 1, or a salt or a tautomer or an isomer thereof, having a structure which is

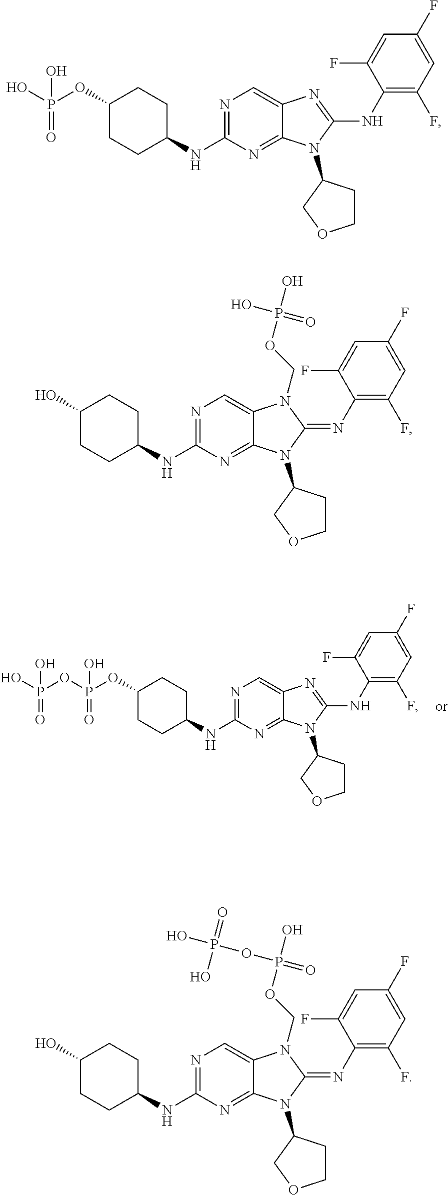 [SMILES: O=P(O)(O)O[C@H]1CC[C@H](Nc2ncc3nc(Nc4c(F)cc(F)cc4F)n([C@H]4CCOC4)c3n2)CC1 ; O=P(O)(O)OCn1/c(=N\c2c(F)cc(F)cc2F)n([C@H]2CCOC2)c2nc(N[C@H]3CC[C@H](O)CC3)ncc21 ; O=P(O)(O)OP(=O)(O)O[C@H]1CC[C@H](Nc2ncc3nc(Nc4c(F)cc(F)cc4F)n([C@H]4CCOC4)c3n2)CC1 ; O=P(O)(O)OP(=O)(O)OCn1/c(=N\c2c(F)cc(F)cc2F)n([C@H]2CCOC2)c2nc(N[C@H]3CC[C@H](O)CC3)ncc21]

18. The compound of claim 1, or a salt or a tautomer or an isomer thereof, having a structure which is

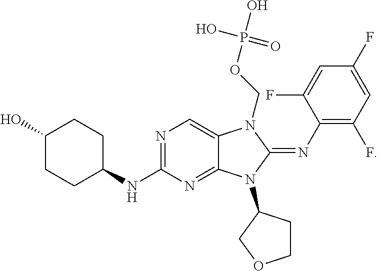 [SMILES: O=P(O)(O)OCn1/c(=N\c2c(F)cc(F)cc2F)n([C@H]2CCOC2)c2nc(N[C@H]3CC[C@H](O)CC3)ncc21]

19. The compound of claim 1, or a salt or a tautomer or an isomer thereof, where the salt is a pharmaceutically acceptable salt.

20. The compound of claim 1, or a salt or a tautomer or an isomer thereof, where the salt of the compound is aluminum, arginine, benzathine, calcium, chloroprocaine, choline, diethanolamine, ethylenediamine, histidine, lithium, lysine, magnesium, meglumine, potassium, procaine, sodium, triethylamine, or zinc, or a combination thereof.

21. The compound of claim 1, or a salt or a tautomer or an isomer thereof, where the salt of the compound is sodium.

22. The compound of claim 1, or a salt or a tautomer or an isomer thereof, which is a disodium salt of:

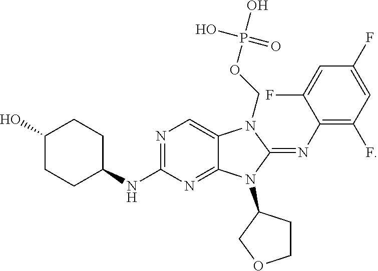 [SMILES: O=P(O)(O)OCn1/c(=N\c2c(F)cc(F)cc2F)n([C@H]2CCOC2)c2nc(N[C@H]3CC[C@H](O)CC3)ncc21]

23. The compound of claim 1, or a salt or a tautomer or an isomer thereof, which is a trisodium salt of:

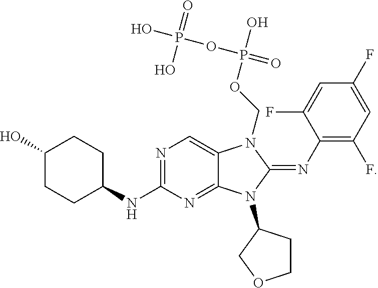 [SMILES: O=P(O)(O)OP(=O)(O)OCn1/c(=N\c2c(F)cc(F)cc2F)n([C@H]2CCOC2)c2nc(N[C@H]3CC[C@H](O)CC3)ncc21]

24. A pharmaceutical formulation comprising:
a) a pharmaceutically acceptable excipient; and
b) the compound, or a pharmaceutically acceptable salt or a tautomer or an isomer thereof, of claim 1.

25. The pharmaceutical formulation of claim 24, wherein the pharmaceutical formulation is an intravenous formulation.

26. A method of treating and/or preventing an injury before, during, or after a surgical procedure to a subject, comprising: administering to the subject a therapeutically and/or prophylactically effective amount of the compound of claim 1, thereby treating and/or preventing the injury before, during, or after the surgical procedure.

27. The method of claim 26, wherein the surgical procedure is on an organ of the subject, and the organ is selected from the group consisting of kidney, liver, heart and brain.

28. The method of claim 26, wherein the surgical procedure is on an organ of the subject, and the organ is selected from the group consisting of kidney, liver, and heart.

29. The method of claim 26, wherein the surgical procedure is on peripheral limb or spinal cord of the subject.

30. The method of claim 26, wherein the injury is ischemia, ischemic reperfusion, embolism, thrombosis, sepsis, toxicity associated with chemotherapy, toxicity associated with radiotherapy, or toxicity associated with a nephrotoxin, or caused by physical trauma, such as being subject to an explosion or being subject to a rapid deceleration event.

31. A method of treating and/or preventing ischemic reperfusion injury to the kidney, lung, heart, brain, or liver before, during, or after a surgical procedure to a subject, comprising: administering to the subject a therapeutically and/or prophylactically effective amount of the compound of claim 1, thereby treating and/or preventing the ischemic reperfusion injury before, during, or after the surgical procedure to a subject.

32. A method of treating and/or preventing ischemic reperfusion injury before, during, or after a surgical procedure to a kidney of a subject, comprising: administering to the subject a therapeutically and/or prophylactically effective amount of a compound having a structure which is: 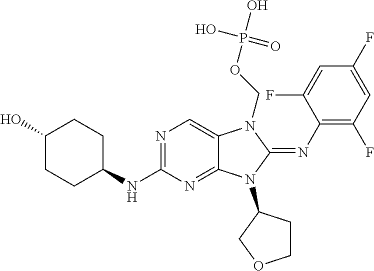 [SMILES: O=P(O)(O)OCn1/c(=N\c2c(F)cc(F)cc2F)n([C@H]2CCOC2)c2nc(N[C@H]3CC[C@H](O)CC3)ncc21]
or a salt or a tautomer or an isomer thereof, thereby treating and/or preventing the ischemic reperfusion injury before, during, or after the surgical procedure to the kidney of the subject.

33. (canceled)

34. (canceled)
